(19) United States
(12) Patent Application Publication
(10) Pub. No.: US 2026/0290550 A1
(43) Pub. Date: Sep. 24, 2026

(54) SYSTEM

(71) Applicant: SoftBank Group Corp., Tokyo (JP)

(72) Inventor: Takuro MORI, Tokyo (JP)

(73) Assignee: SoftBank Group Corp., Tokyo (JP)

(21) Appl. No.: 19/568,865

(22) Filed: Mar. 17, 2026

(30) Foreign Application Priority Data
Mar. 19, 2025 (JP) 2025-045180

(51) Int. Cl. G16H 20/60; A61B 5/00; G06F 40/205; G06F 40/284; G06V 10/26; G06V 10/764; G06V 10/82; G06V 40/16; G10L 25/63

(52) U.S. Cl. CPC G16H 20/60; A61B 5/6803; G06F 40/205; G06F 40/284; G06V 10/26; G06V 10/764; G06V 10/82; G06V 40/174; G10L 25/63

(57) ABSTRACT

A system includes a processor that is configured to collect information on leftover food using one or more sensors, acquire information on a health status of a user from a wearable device, acquire information on prices and freshness of grocery items from an online database, recognize an emotional state of the user using at least one of a camera and a microphone, generate a prompt that instructs a generative AI model to generate an optimal meal plan and an optimal shopping list based on analysis of the collected information, cause the generated meal plan and the generated shopping list to be displayed through a user interface, and arrange delivery of food from a partner restaurant via an online ordering system.

### CROSS-REFERENCE TO RELATED APPLICATION

This application is based on and claims priority under 35 USC 119 from Japanese Patent Application No. 2025-045180 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional meal planning and grocery recommendation systems are limited in their ability to holistically reflect a user's real-world context, such as actual leftover food in the household, current health status, emotional condition, and dynamic market information including prices and freshness of grocery items. Many existing systems rely primarily on static user input, generic recipe databases, or simple rule-based recommendations, and therefore fail to sufficiently reduce food waste, to support health-oriented objectives such as dieting or stress reduction, and to optimize cost performance across multiple stores. Furthermore, known systems typically do not leverage generative AI models through contextually appropriate prompts that integrate multimodal data from sensors, cameras, microphones, wearable devices, and online databases. As a result, users are often required to manually coordinate meal planning, shopping list generation, and optional food delivery from restaurants, which is time-consuming, cognitively burdensome, and prone to suboptimal nutritional and economic outcomes. In addition, existing systems rarely adapt the content or strategy of meal and shopping suggestions to specific user objectives, such as targeted weight control or mental well-being, while simultaneously coordinating in-home food utilization with external food delivery services. Accordingly, there is a need for a system that can automatically collect and integrate diverse user-related and environment-related information, prompt a generative AI model in a flexible manner tailored to various user goals, generate optimal meal plans and shopping lists, and, when appropriate, coordinate delivery of food from partner restaurants, thereby reducing food waste, improving health support, and enhancing cost-efficiency and user convenience.

### SUMMARY

In order to solve the above-described problems, according to one aspect of the present invention, there is provided a system comprising a processor, wherein the processor is configured to collect information on leftover food using one or more sensors, acquire information on a health status of a user from a wearable device, acquire information on prices and freshness of grocery items from an online database, recognize an emotional state of the user using at least one of a camera and a microphone, generate a prompt that instructs a generative AI model to generate an optimal meal plan and an optimal shopping list based on analysis of the collected information, cause the generated meal plan and the generated shopping list to be displayed through a user interface, and arrange delivery of food from a partner restaurant via an online ordering system. In one embodiment, the processor is further configured to propose the optimal meal plan and the optimal shopping list so as to cover various patterns by using prompts corresponding to specific objectives including at least one of dieting and stress reduction, thereby enabling the generative AI model to tailor its outputs to diverse user goals. In another embodiment, the processor is further configured to propose optimal shopping using information on a plurality of stores acquired from the online database so as to identify inexpensive and high-quality products, and to arrange delivery of food from the partner restaurant via the online ordering system, thereby allowing the system to optimize both in-store purchases and outsourced food provision. Through these functions, the system integrates multimodal sensing, health and emotional state analysis, dynamic market data, and generative AI prompt engineering to automatically produce and present context-aware, goal-oriented meal plans and shopping lists, while also coordinating restaurant food delivery, thereby achieving reduction of food waste, improvement of health support, enhancement of cost performance, and reduction of user effort.

The term “processor” refers to any hardware or combination of hardware and software that executes instructions to perform the functions recited in the claims, including but not limited to a central processing unit (CPU), a microcontroller, a digital signal processor, a graphics processing unit (GPU), an application-specific integrated circuit (ASIC), a field-programmable gate array (FPGA), or a distributed set of such components. The term “sensor” refers to any device or module that detects or measures a physical quantity related to food items and converts it into a signal or data, including but not limited to weight sensors, temperature sensors, humidity sensors, proximity sensors, radio-frequency identification (RFID) readers, barcode readers, and image capture devices used for identifying leftover food.

The term “leftover food” refers to any food items, ingredients, or prepared dishes that remain available for consumption after previous meals or usage, and that are stored in a household or user environment, such as in a refrigerator, pantry, cupboard, or similar storage location.

The term “wearable device” refers to any electronic device designed to be worn by the user and capable of measuring or estimating physiological, behavioral, or environmental parameters, including but not limited to smartwatches, fitness bands, smart rings, smart glasses, or health-monitoring patches.

The term “health status” refers to information indicative of the physical or physiological condition of the user, including but not limited to body weight, heart rate, blood pressure, activity level, sleep patterns, caloric expenditure, dietary restrictions, allergies, and chronic medical conditions.

The term “online database” refers to any remotely accessible data storage system or service available over a network, including but not limited to cloud-based databases, web services, and application programming interfaces (APIs), from which information such as product prices, freshness data, and store inventories can be obtained.

The term “prices and freshness of grocery items” refers to information describing economic and quality attributes of food products available from grocery stores, including but not limited to unit price, discount information, stock availability, expiration date, best-before date, harvest date, shelf life, freshness score, or quality rating.

The term “emotional state of the user” refers to information indicative of the user's affective or psychological condition at a given time, including but not limited to states such as stress, relaxation, happiness, sadness, frustration, or fatigue.

The term “camera” refers to any image capture device capable of obtaining still images or video of the user or the environment, including but not limited to built-in cameras in smartphones, tablets, laptops, dedicated webcams, or surveillance cameras.

The term “microphone” refers to any audio capture device capable of acquiring sound signals from the user or the environment, including but not limited to built-in microphones in smartphones, headsets, smart speakers, or standalone recording devices.

The term “generative AI model” refers to any artificial intelligence model capable of generating output data such as text, images, or other content in response to input data or prompts, including but not limited to large language models, multimodal foundation models, and specialized generative models for recipes or shopping lists.

The term “prompt” refers to any data structure, message, instruction, or set of parameters provided as input to the generative AI model that specifies context, constraints, objectives, or desired output format for generating the meal plan and the shopping list.

The term “optimal meal plan” refers to a set of one or more meal proposals generated by the system that are determined, according to one or more criteria, to be favorable for the user, including but not limited to criteria relating to nutritional balance, caloric targets, health objectives, user preferences, emotional state, available leftovers, cost, and preparation complexity.

The term “optimal shopping list” refers to a list of items to be purchased that is determined, according to one or more criteria, to be favorable for the user, including but not limited to criteria relating to total cost, product quality, freshness, store availability, compatibility with the optimal meal plan, and reduction of food waste.

The term “user interface” refers to any hardware and software components that enable interaction between the user and the system, including but not limited to graphical user interfaces on smartphones, tablets, or computers, voice-based interfaces through smart speakers, and mixed or augmented reality interfaces.

The term “online ordering system” refers to any electronic commerce platform or service that enables the user or the system to place orders for food or related products over a network, including but not limited to web-based ordering systems, mobile ordering applications, and integrated APIs of food delivery services.

The term “partner restaurant” refers to any restaurant, food service provider, or similar entity that has a cooperative or contractual relationship with the system operator, and that accepts orders placed via the online ordering system for preparing and delivering food to the user.

The term “delivery of food” refers to the process of preparing, packaging, and transporting food from a partner restaurant or similar provider to a location designated by the user, such as the user's home or workplace.

The term “specific objectives” refers to user-oriented goals or purposes that influence the content or strategy of meal and shopping suggestions, including but not limited to dieting, weight control, stress reduction, mental well-being, muscle gain, or management of particular health conditions.

The term “various patterns” refers to different combinations or scenarios of meal plans and shopping lists that correspond to different specific objectives, user preferences, schedules, and consumption habits, such that the system can flexibly adapt its recommendations to diverse user needs.

The term “plurality of stores” refers to two or more distinct retail entities or sales channels, including physical grocery stores, online grocery services, and hybrid models, from which price and freshness information can be obtained and among which comparisons can be made.

The term “inexpensive and high-quality products” refers to grocery items that, when evaluated according to predetermined criteria, provide favorable cost-to-quality or cost-to-freshness characteristics, such that the user can obtain economic benefits without sacrificing desired quality or freshness levels.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

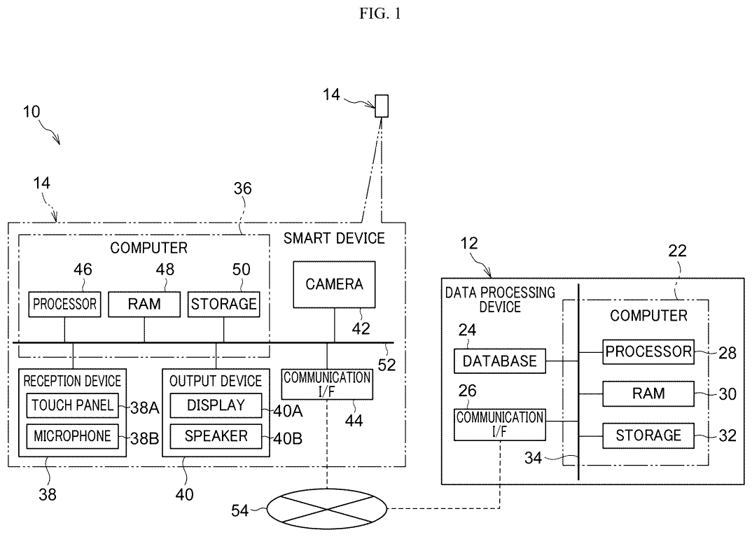

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

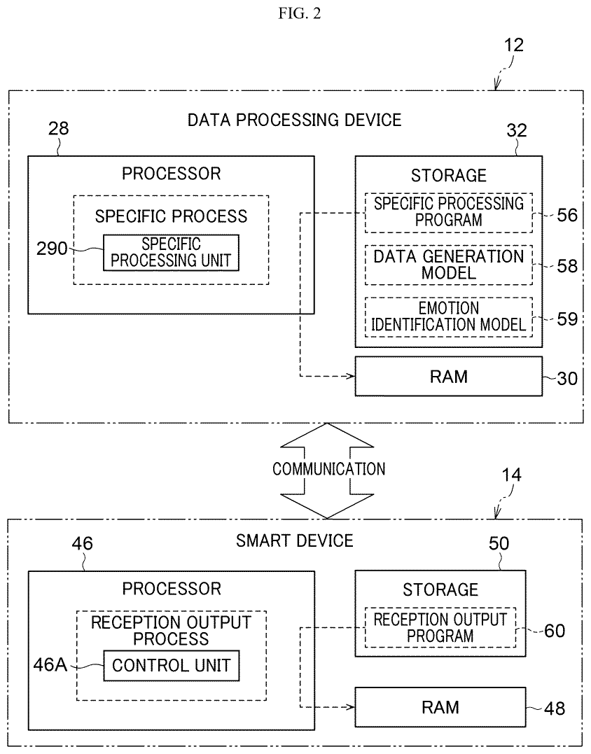

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented meal planning and grocery recommendation systems typically rely on rule-based engines or simple optimization algorithms that process limited categories of input data, such as static recipes or basic price lists. Such systems are not designed to integrate heterogeneous, dynamically changing data sources, including fine-grained food inventory captured by sensors and cameras, detailed physiological and behavioral data from wearable devices, and multi-store price and quality information obtained from network resources. As a result, existing systems often generate recommendations that are either generic, nutritionally suboptimal, economically inefficient, or poorly aligned with user preferences and constraints.

In particular, there are several technical problems. First, there is no efficient computer mechanism to transform raw, multimodal data streams (sensor readings, image data, health metrics, and network-sourced commerce data) into a coherent, machine-usable representation that can be effectively consumed by a generative AI model. Absent such a mechanism, the generative AI model may receive incomplete or unstructured context, leading to unstable output quality and unpredictable behavior.

Second, conventional systems lack a structured, programmatic way to construct and iteratively refine natural-language prompt sentences for a generative AI model based on user interaction and historical preference data. Without automated prompt construction and feedback integration, the system cannot adapt its generative behavior to changing nutritional goals, cost constraints, and user preferences, resulting in inefficient use of computing resources and repeated manual tuning.

Third, existing architectures do not provide a tightly integrated pipeline that post-processes the free-form output of a generative AI model into structured data, verifies compliance with user-specific constraints (for example, allergies, calorie limits, or store preferences), and then feeds this verified structure back into subsequent prompt sentences. This lack of a closed feedback loop leads to inconsistent recommendations, increases the need for manual corrections by the user, and reduces the reliability and scalability of the overall computing system.

Fourth, there is no unified, computer-implemented optimization framework that combines generative content creation with quantitative cost and quality indices across multiple sales facilities to propose economically efficient purchasing routes. Traditional price comparison mechanisms are separated from the content generation process and do not influence the generative model's internal decision-making in a fine-grained and iterative manner. Therefore, there is a need for a computer technology that improves how a server acquires heterogeneous input data, generates and refines prompt sentences for a generative AI model, converts the model's output into verifiable structured information, enforces user-specific constraints automatically, and integrates cost and quality optimization for multiple vendors. Such an improved architecture should enhance the accuracy, personalization, and computational efficiency of meal planning and shopping list generation, thereby improving overall computer system performance and user experience.

The specific processing by the specific processing unit 290 of the data processing device 12 in Example 1 is realized by the following means.

The present invention provides a server comprising a processor configured to acquire inventory information of food stored in a storage apparatus by using a sensor device and an imaging device, acquire health information including physiological information and behavioral information of a user from a biological information acquisition device, acquire price information and quality information relating to a plurality of sales facilities via a communication network, generate planning target information including a nutritional goal and preference conditions of the user on the basis of the inventory information, the health information, the price information, and the quality information, construct the planning target information as a natural-language prompt sentence, input the prompt sentence to a generative AI model to cause the generative AI model to generate a meal plan for a predetermined period and a shopping list including purchase candidates for the plurality of sales facilities, analyze the meal plan and the shopping list obtained from the generative AI model, convert the meal plan and the shopping list into structured information including items, quantities, nutritional values, and candidate sales facilities, when a component that violates a constraint condition of the user is detected, correct the meal plan and the shopping list by removing or replacing the component, cause a user interface device to display the meal plan and the shopping list on the basis of the structured information, acquire preference information and modification operations from the user via the user interface device, update the planning target information and the prompt sentence based on the preference information and the modification operations, execute a regeneration process for the generative AI model by using the updated prompt sentence so as to iteratively improve the meal plan and the shopping list in accordance with the preference information of the user, and arrange delivery of products or prepared food via an online transaction processing apparatus on the basis of the shopping list and the price information and the quality information relating to the plurality of sales facilities. This enables the server to implement an improved, closed-loop computing pipeline in which heterogeneous sensor, health, and commerce data are unified into optimized natural-language prompts, the generative AI model is guided and refined through structured feedback and constraint enforcement, and the resulting meal plans and shopping routes are automatically optimized for nutritional suitability, user preference, and cost-effectiveness, thereby enhancing the technical performance, reliability, and efficiency of the computer-based recommendation system.

The term “sensor device” refers to a hardware component or a combination of hardware components configured to detect a physical property related to stored food, such as weight, presence, temperature, or identification information, and to output corresponding digital signals to the processor.

The term “imaging device” refers to an electronic image capture component, such as a camera or an array of cameras, configured to acquire digital image data representing the interior of a storage apparatus and food contained therein.

The term “storage apparatus” refers to an equipment unit or installation configured to store food under controlled or uncontrolled environmental conditions, including but not limited to a refrigerator, a freezer, a pantry, or a cabinet.

The term “inventory information” refers to data representing types, quantities, and optionally storage states or expiration-related attributes of food items present in a storage apparatus at a given time.

The term “biological information acquisition device” refers to an electronic device worn on or attached to the body of a user, or otherwise configured to measure physiological or behavioral parameters of the user, such as heart rate, body weight, physical activity level, or sleep pattern, and to output corresponding digital data.

The term “health information” refers to data describing a physiological state and a behavioral state of a user, including at least one of heart rate, weight, activity level, energy expenditure, and similar metrics, optionally combined with user-specific constraints such as dietary restrictions or medical conditions.

The term “communication network” refers to a wired or wireless data communication infrastructure, such as a local network, a wide area network, or the Internet, that enables data exchange between the server, external data sources, and user interface devices. The term “sales facility” refers to a commercial entity providing products or prepared food for purchase, including but not limited to a physical retail store, an online store, or a food service provider.

The term “price information” refers to data representing monetary cost or related pricing attributes of products or prepared food offered by a sales facility, including base price, discount price, unit price, or price per quantity.

The term “quality information” refers to data indicating a qualitative characteristic of products or prepared food, such as freshness, rating, review score, or other quality-related indicators associated with a sales facility.

The term “planning target information” refers to an internal data structure generated by the processor that combines inventory information, health information, price information, quality information, and constraint information, and that represents conditions and objectives for generating a meal plan and a shopping list.

The term “nutritional goal” refers to one or more quantitative or qualitative targets related to nutrient intake for a user, including but not limited to total calorie intake, macronutrient balance, or specific nutrient limits or requirements.

The term “preference conditions” refers to data expressing a user's likes, dislikes, dietary style, ingredient preferences, cuisine preferences, or other user-specific selection tendencies that influence generation of a meal plan and a shopping list.

The term “prompt sentence” refers to a sequence of natural-language text constructed by the processor that encodes planning target information and instructions, and that is used as input to a generative AI model to control and constrain the model's output.

The term “generative AI model” refers to a machine learning model, such as a neural network-based language model, configured to generate natural-language text or structured content in response to an input prompt sentence.

The term “meal plan” refers to information representing a schedule or collection of proposed meals for one or more time periods, including identification of dishes, ingredients, and optionally nutritional properties for each proposed meal.

The term “shopping list” refers to information representing a collection of products or ingredients to be purchased, including item identifiers, quantities, and optionally recommended sales facilities for obtaining such items.

The term “structured information” refers to data that is organized in a predefined format, such as records, fields, arrays, or key-value pairs, enabling programmatic access to elements including items, quantities, nutritional values, and candidate sales facilities.

The term “constraint condition” refers to a rule or limitation associated with a user or system operation, such as an allergy, an ingredient prohibition, a calorie limit, a budget limit, a time constraint, or a store exclusion.

The term “user interface device” refers to an electronic device configured to present information to a user and receive input from the user, such as a mobile terminal, a tablet, a personal computer, or a smart display.

The term “preference information” refers to data obtained from user actions or explicit inputs indicating acceptance, rejection, modification, or prioritization of specific meals, ingredients, stores, or other elements of a generated plan.

The term “modification operations” refers to user-performed actions via a user interface that change a generated meal plan or shopping list, such as adding, removing, or substituting items, altering quantities, or changing selected sales facilities.

The term “regeneration process” refers to a computational procedure in which the processor updates a prompt sentence based on new or modified planning target information and causes the generative AI model to generate a revised meal plan and shopping list.

The term “online transaction processing apparatus” refers to a server or system configured to execute purchase-related operations via a communication network, including order placement, payment processing, and delivery arrangement for products or prepared food.

The term “purpose information” refers to data describing one or more high-level objectives of a user or system, such as weight reduction, nutritional balance improvement, mental load reduction, time saving, or cost reduction, that guide generation of a meal plan and a shopping list.

The term “historical preference information” refers to data derived from past user interactions, selections, or feedback related to previously generated meal plans or shopping lists, and used to adjust future generation processes.

The term “cost index” refers to a computed numerical value representing a relative or absolute cost characteristic of an item at a sales facility, based on price information and optionally on quantity or unit size.

The term “quality index” refers to a computed numerical value representing a quality characteristic of an item at a sales facility, based on quality information such as freshness, ratings, or other quality-related parameters.

The term “purchasing route” refers to a sequence or combination of sales facilities and associated items to be purchased, determined in such a way as to satisfy a meal plan and shopping list while optimizing at least one of cost efficiency and quality.

In one embodiment, a server executes a computer program that integrates heterogeneous data sources, constructs natural-language prompt sentences, invokes a generative AI model, and post-processes model outputs into structured, constraint-compliant plans. The server cooperates with a terminal operated by a user, and with physical devices such as a storage apparatus equipped with sensors and an imaging device, and a biological information acquisition device.

The server uses general-purpose computing hardware including at least one central processing unit, a system memory, non-volatile storage, and a network interface. In some embodiments, the server further uses an accelerator device such as a graphics processing unit or a tensor-processing unit to perform inference of a neural network-based generative AI model. The server executes software components including an operating system, a web application framework, a database management system, and machine learning libraries. For example, the server runs a Linux-based operating system, a web framework implemented in a scripting language (such as a Python-based or JavaScript-based framework), a relational or document-oriented database (such as a SQL-based database or a NoSQL database), and a machine learning library such as a tensor computation library or a deep learning framework for neural network inference.

The server acquires inventory information by communicating with a storage apparatus such as a refrigerator. The storage apparatus includes at least one sensor device and at least one imaging device. The sensor device may be a weight sensor, a temperature sensor, or a radio-frequency identification reader. The imaging device may be a digital camera. The server receives image data and sensor readings via a local network or a wide area network. The server uses an image processing library, such as a general-purpose computer vision library, to perform preprocessing operations including resizing, normalization, and color space conversion on the acquired image data. The server then uses an image recognition model implemented in a neural network framework, such as a convolutional neural network with multiple convolutional layers, pooling layers, and fully connected layers, to classify visual objects corresponding to stored food items and to estimate item boundaries. The server associates the classification results with sensor readings to infer item types, estimated quantities, and optional expiration-related attributes. The server stores the resulting inventory information in a structured data format in a database table dedicated to inventory records.

The server acquires health information by receiving data from a biological information acquisition device via the terminal. The terminal is a user-operated computing device such as a smartphone or a tablet. The terminal executes an application that communicates with an application programming interface of the biological information acquisition device, such as a wearable health tracker or a smart scale. The terminal reads health metrics including heart rate, body weight, step count, energy expenditure, and similar parameters. The terminal aggregates these metrics over a predefined period and transmits them to the server as structured data via a secure network protocol. The server validates and parses the received health information, stores it in a database table for user health, and computes derived parameters such as body mass index, average daily activity level, and average resting heart rate. The server thereby maintains an updated health profile for each user.

The server acquires price information and quality information from a plurality of sales facilities through a communication network. The server executes a network access module that sends requests to remote servers of online stores or service providers. The server retrieves data such as item identifiers, base prices, discounted prices, unit sizes, freshness indicators, rating values, and review-derived quality metrics. The server normalizes pricing into comparable values, such as price per unit mass or price per piece, and constructs quality indices by combining freshness measures and rating values. The server stores these values in database structures keyed by item type and sales facility identifier.

The server generates planning target information by combining inventory information, health information, price information, quality information, and stored user preference information.

The server maintains preference information as records indicating user likes and dislikes for specific ingredients, cuisine types, preparation methods, and sales facilities. The server also maintains constraint information such as allergies, prohibited ingredients, maximum daily calorie intake, and budget limits. The server constructs a planning target object that includes nutritional goals, such as a target daily energy intake and macronutrient ratios; preference conditions, such as favored cuisine styles and ingredients to avoid; and economic objectives, such as cost reduction and quality thresholds. The server serializes this internal object into a natural-language prompt sentence.

The server constructs the prompt sentence using template-based generation and rule-based enrichment, rather than simply concatenating raw data. The server uses language templates that include explicit sections for inventory status, health status, nutritional goal, cost and quality information, user preferences, and explicit instructions to the generative AI model regarding output structure. By using a deterministic template system, the server reduces ambiguity and variance in the instructions provided to the generative AI model, thereby improving the stability and predictability of model outputs.

For example, the server generates a prompt sentence such as:
“The user is currently on a diet and wants to limit daily intake to about 1,600 kcal. The refrigerator inventory includes 300 g of chicken breast, 2 tomatoes, 1 head of lettuce, 4 eggs, and 200 g of yogurt. The user's wearable device data show a moderate to high activity level and no specific medical restrictions except a preference for low-fat, high-protein meals. Current store data indicate that Store A offers the lowest prices for chicken breast, tomatoes, and lettuce while maintaining good freshness and quality ratings. Please propose a 3-day meal plan that uses the existing ingredients as efficiently as possible, focuses on low-calorie, high-protein dishes, and stays close to 1,600 kcal per day. Also, create a detailed shopping list specifying which additional items to buy, at which store, and in what quantities, in order to follow the proposed meal plan at the lowest total cost.”

In another example, after the user has provided feedback, the server generates a modified prompt sentence such as:
“User rejected meals containing broccoli and requested spicier dishes. Considering the updated preference to avoid broccoli and prefer spicy flavor profiles, and given the same inventory and current store prices, please regenerate a 2-day dinner plan and an optimized shopping list, clearly marking recommended stores for each ingredient.”

The server inputs the prompt sentence into a generative AI model. In some embodiments, the generative AI model is a transformer-based language model implemented using a deep learning framework. The model includes an embedding layer that converts tokens of the prompt sentence into dense vectors, a multi-layer stack of self-attention and feed-forward blocks, and an output layer that generates probabilities over a vocabulary of tokens. The server executes the generative AI model on a hardware accelerator to perform parallel matrix multiplications and attention weight calculations. The server may use a pre-trained model fine-tuned on domain-specific text, such as recipe collections, nutritional guidelines, and shopping list examples.

The server performs training or fine-tuning of the generative AI model by using supervised learning and, optionally, reinforcement learning based on user feedback. During training, the server computes a loss function such as cross-entropy loss between predicted tokens and target tokens. The server updates model parameters by performing gradient-based optimization, such as stochastic gradient descent or an adaptive optimization algorithm. The server applies regularization techniques and data augmentation, such as paraphrasing of instructions and diversification of ingredient lists, to improve generalization and robustness. By tailoring the training process to structured meal and shopping plan outputs, the server configures the model so that its internal parameters capture patterns relevant to nutritional planning and cost optimization, thereby improving the accuracy and efficiency of the generative process.

The server executes inference by selecting sampling parameters such as temperature, top-k, or top-p thresholds to control diversity of generated text. The server imposes internal constraints, such as maximum token lengths for the meal plan section and the shopping list section, to ensure that outputs are computationally manageable and suitable for subsequent parsing.

The server receives the generated text from the generative AI model. The server then applies a post-processing module to convert the generated text into structured information. The server uses pattern matching, tokenization, and rule-based segmentation to identify meal headers, ingredient lines, quantity expressions, and store annotations. For example, the server detects patterns like “Day 1—Breakfast,” “Ingredients: 150 g chicken breast, 1 tomato,” and “Buy at Store A.” The server maps these patterns into records with fields such as date, meal type, dish name, ingredient identifier, quantity, unit, and store identifier. The server stores the structured information in database tables and in memory-resident data structures for fast access.

The server verifies that the structured information satisfies constraint conditions. The server computes estimated nutritional values using a nutrition database, for example a remote or local food composition database. The server calculates total daily calories and macronutrient distribution for each day of the meal plan. The server checks allergen lists and prohibited ingredients, and identifies any conflicts. If a violating component is detected, the server removes or replaces the corresponding ingredient or dish. In some embodiments, the server uses deterministic substitution rules, such as replacing a prohibited ingredient with a nutritionally similar alternative. In other embodiments, the server regenerates parts of the plan by constructing an updated prompt sentence that explicitly describes the violation and required constraints.

The server computes cost indices and quality indices for each item in the shopping list. The server retrieves relevant price and quality records for each item across sales facilities and calculates a cost index, such as price per unit, and a quality index, such as a normalized combination of freshness and rating. The server selects a preferred sales facility for each item by optimizing at least one of the cost index and the quality index. The server incorporates the selected facility information into the structured shopping list and, when appropriate, into subsequent prompt sentences. Because the server feeds back quantitative optimization results into prompt sentences, the generative AI model can adapt its content generation in light of actual cost and quality trade-offs, rather than operating solely in an abstract recipe space. The server communicates the structured meal plan and shopping list to the terminal. The terminal displays the information via a graphical user interface. The terminal may present daily or weekly views, allow filtering by meal type, and display cost summaries per store. The terminal enables the user to perform modification operations such as removing a dish, substituting an ingredient, approving a suggested store, or changing a quantity. The terminal transmits these user inputs to the server as preference information and modification operations.

The server updates user preference information based on the received feedback. The server maintains statistics, such as counts of how often a user accepts or rejects certain ingredients, dishes, or cuisine styles. The server incorporates this historical preference information into future planning target information and future prompt sentences. For example, the server may attach a statement such as “The user prefers spicy food and dislikes broccoli. Avoid broccoli and increase the use of chili-based seasonings.” The server thus performs a closed-loop adaptation process, in which prompt sentences and model behavior are progressively refined by quantifiable user feedback and model performance metrics.

The server arranges delivery of products or prepared food via an online transaction processing apparatus. The server assembles order data including selected items, quantities, selected sales facilities, delivery addresses, and payment-related tokens. The server transmits this information to the transaction processing apparatus using a defined application programming interface. The transaction processing apparatus executes order placement and payment authorization steps. By automating the linkage between optimized shopping lists and transaction execution, the server reduces repetitive data entry and lowers the risk of human error during order placement.

The described architecture improves computer technology in several ways. The server uses a specific data structure for planning target information and a structured prompt-generation algorithm that transforms heterogeneous sensor data, health metrics, and store information into well-formed natural-language instructions for a generative AI model. This transformation increases the information density and coherence of model input, which reduces the number of tokens and rounds of inference required to obtain an acceptable plan, thereby improving computational efficiency and reducing network and processing load.

The server performs a specialized post-processing and validation pipeline that converts free-form model output into verifiable structured information, automatically detects constraint violations, and applies deterministic corrections or guided regeneration. This pipeline reduces the need for manual review and correction by the user, and it enhances the reproducibility and reliability of AI-generated content. Because the server explicitly decomposes and normalizes generated text into machine-readable fields, the system supports advanced indexing, caching, and incremental updates, which improve data management and retrieval efficiency.

The server implements a closed feedback loop between user preference information and model prompting. This loop is realized by continuously updating the planning target information and prompt sentences, rather than relying on static instructions. This continuous adaptation changes how the computing system allocates computational resources, focusing generative capacity on regions of the solution space that are statistically more relevant to the particular user. As a result, the server reduces wasted computation on unlikely or undesirable plans and improves convergence toward user-acceptable solutions.

The server further integrates optimization of cost and quality indices into both the structured shopping list and the generative prompt content. This integration differs from simple post-hoc price sorting because the optimization results influence the generative process itself. By including explicit cost and quality summaries in prompt sentences, the server guides the generative AI model toward plans that are not only nutritionally appropriate but also aligned with actual market conditions. This interaction yields technical effects such as fewer regeneration cycles, reduced token generation, and lower network traffic for repeated corrections.

The use of a transformer-based generative AI model with explicit training on structured meal and shopping data, combined with the described prompt-construction and post-processing modules, provides an AI system that operates according to non-conventional, computer-specific rules. The server employs particular tokenization strategies, loss functions, and reinforcement signals to shape the model's internal representation. The combination of: (i) sensor-driven inventory classification, (ii) health-profile aggregation, (iii) multi-store cost and quality optimization, (iv) template-based prompt construction, and (v) structured post-processing and closed-loop refinement, yields a new computing architecture that improves processing speed, output accuracy, and resource utilization relative to conventional rule-based recommendation engines or generic text generation systems.

The terminal and the user cooperate with the server in this architecture. The user provides explicit feedback through the terminal regarding acceptance or modification of generated plans. The terminal operates as an interactive control surface that supplies high-quality training signals to the preference model maintained by the server. This interaction allows the server to refine internal weighting of features, such as the importance of specific dietary goals versus cost minimization, which further improves calculation efficiency and accuracy for future planning sessions.

Alternative embodiments are possible within the same inventive concept. In some embodiments, the server executes the generative AI model locally on an accelerator device; in other embodiments, the server calls an external model endpoint via a network. In some embodiments, the image recognition components use a convolutional neural network; in other embodiments, a vision transformer architecture or a hybrid model is used. In some embodiments, the nutrition estimation module queries an external nutrition database; in other embodiments, the server stores a local cache of nutritional values for faster response. In further embodiments, the terminal is implemented as a web browser on a personal computer, while in other embodiments it is a native application on a mobile device or a smart display. Across these variations, the common technical features include automated acquisition of heterogeneous data, structured construction of prompt sentences, transformer-based generative inference, deterministic post-processing to structured form, constraint enforcement, closed-loop feedback integration, and cost and quality optimization across multiple sales facilities. Collectively, these features implement a computer-based system that goes beyond generic automation of human tasks and instead improves fundamental aspects of data processing, model interaction, and computational efficiency in the context of generative meal planning and shopping list generation.

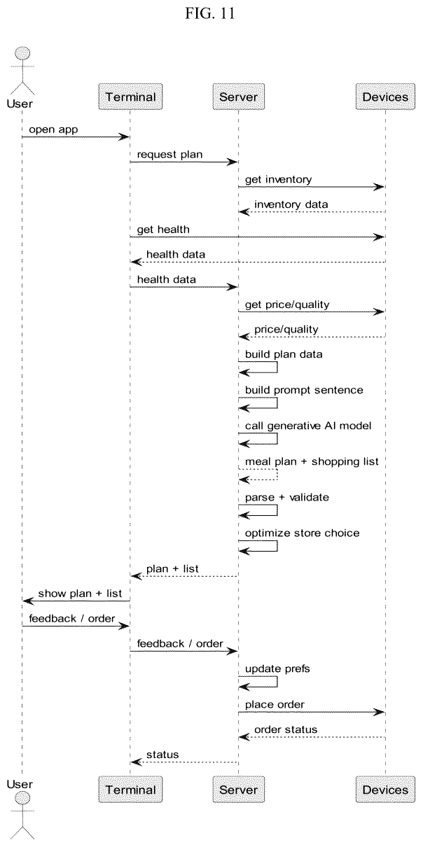

The following describes the processing flow using FIG. 11.

### Step 1:

Server acquires raw inventory data from a storage apparatus.

Server receives, as input, image data captured by an imaging device inside a storage apparatus and sensor readings from one or more sensor devices (for example, weight sensors or identification readers). Server applies image preprocessing operations, including resizing, normalization, and noise reduction, to convert each raw image into a standardized tensor representation. Server then executes an image recognition algorithm, such as a convolutional neural network, on the preprocessed image to classify visible objects into food categories and to localize their positions. Server combines the classification results with sensor readings by matching time stamps and device identifiers, and performs data fusion to estimate item types, quantities, and optional expiration-related attributes. As output, server generates structured inventory records and stores them in an inventory table in a database.

### Step 2:

Terminal acquires health information from a biological information acquisition device and transmits it to server.

Terminal receives, as input, raw physiological and behavioral data from a wearable or other biological information acquisition device via a local wireless interface such as Bluetooth or a short-range wireless protocol. Terminal aggregates time-series measurements of heart rate, body weight, step count, and activity level into summary statistics, such as daily averages, maximum values, and total steps. Terminal formats the aggregated data into a structured representation, for example a JSON object including user identifier, measurement period, and aggregated metrics. Terminal sends this structured health information to server over a secure network connection. As output, terminal provides a validated health data package to server for further processing.

### Step 3:

Server processes health information and updates user health profiles.

Server receives, as input, the structured health data package transmitted by terminal. Server validates the data by checking required fields, data types, and value ranges, and discards or flags inconsistent records. Server computes derived health metrics, such as body mass index from weight and height, average daily energy expenditure from activity level, and resting heart rate from low-activity periods. Server then updates a user health profile table in the database by inserting new records or modifying existing ones associated with the corresponding user identifier. As output, server maintains an up-to-date health profile that can be queried during subsequent planning operations.

### Step 4:

Server acquires price and quality information from multiple sales facilities.

Server receives, as input, configuration data defining a list of target sales facilities, product categories of interest, and network addresses of remote data sources. Server sends network requests, such as HTTP requests, to remote servers operated by sales facilities or aggregators and retrieves response data containing item names, prices, discounts, unit sizes, freshness scores, and user ratings. Server parses the response payloads, which may be in structured formats such as JSON or HTML, to extract relevant fields. Server normalizes prices to a common unit (for example, price per 100 g or price per unit) and computes quality indices by combining freshness scores and ratings using a predefined formula. As output, server generates and stores price and quality records keyed by item and sales facility identifiers in a database.

### Step 5:

Server computes cost indices and quality indices for items relevant to the user's inventory and health profile.

Server receives, as input, the current inventory records, the updated health profile, and the raw price and quality records for a broad set of items. Server first identifies candidates relevant to the user by matching inventory items and potential complementary ingredients against available products at sales facilities. Server then calculates a cost index for each candidate item by dividing normalized price by unit quantity and optionally incorporating discount factors. Server calculates a quality index by applying a weighted sum or other aggregation method to freshness and rating values. Server stores these computed indices in a data structure that links each item to one or more sales facilities. As output, server provides a set of cost and quality indices that will influence subsequent planning and prompt construction.

### Step 6:

Server generates planning target information based on unified data.

Server receives, as input, inventory records, the health profile, computed cost and quality indices, and stored user preference and constraint information. Server applies rule-based logic to determine nutritional goals (for example, daily calorie targets and macronutrient ratios) from the health profile and user-specified objectives such as weight reduction or performance improvement. Server filters ingredients according to constraint conditions, such as allergens and prohibited ingredients, by cross-referencing ingredient identifiers with a constraint list.

Server then constructs a planning target object that includes fields for nutritional goals, inventory status, permissible and preferred ingredients, candidate items with cost and quality indices, and economic objectives such as budget limits. As output, server produces an internal, structured planning target representation that will be converted into a prompt sentence.

### Step 7:

Server constructs a natural-language prompt sentence for the generative AI model.

Server receives, as input, the planning target object produced in Step 6. Server selects appropriate language templates depending on the planning horizon (for example, number of days), the type of meals being planned (such as dinners only or all daily meals), and the optimization focus (cost, quality, or a combination). Server then fills template placeholders with specific values from the planning target object, including current inventory quantities, nutritional goals, cost and quality summaries, and user preferences. Server also inserts explicit instructions regarding output format, such as separate sections for “Meal Plan” and “Shopping List,” and constraints such as maximum daily calories. Server concatenates these elements into a coherent natural-language text string. As output, server generates a prompt sentence that encodes the planning target information and guides the generative AI model.

### Step 8:

Server invokes the generative AI model and obtains a candidate meal plan and shopping list.

Server receives, as input, the constructed prompt sentence. Server tokenizes the prompt sentence into tokens using a tokenizer associated with the generative AI model, and converts tokens into embeddings through an embedding layer. Server executes the generative AI model, which may be a transformer-based neural network with multiple self-attention layers and feed-forward layers, to compute attention weights and propagate activations through the network. Server applies a decoding strategy, such as greedy decoding or top-k sampling, to generate successive output tokens until an end condition is satisfied. Server concatenates the generated tokens into text segments, which form a proposed meal plan and a shopping list following the requested structure. As output, server obtains a raw, textual candidate meal plan and shopping list from the generative AI model.

### Step 9:

Server converts the generated text into structured information and validates constraint compliance.

Server receives, as input, the raw text output of the generative AI model from Step 8. Server applies a parsing module that uses pattern matching, regular expressions, and text segmentation rules to identify segment boundaries such as days, meal types, ingredient lines, and store recommendations. Server extracts entities including dish names, ingredient identifiers, quantities, units, and associated sales facilities, and maps these entities to standardized codes or identifiers stored in the database. Server then computes estimated nutritional values for each meal by querying a nutrition database and summing the nutrient contributions of individual ingredients. Server checks each meal and the overall plan against user constraint conditions, such as maximum daily calories and prohibited ingredients, by comparing extracted entities and nutrient values to constraint lists and thresholds. As output, server generates a structured, constraint-checked representation of the meal plan and shopping list.

### Step 10:

Server corrects violations and, if needed, regenerates portions of the plan.

Server receives, as input, the structured meal plan and shopping list along with flags indicating any detected violation of constraint conditions. Server identifies specific components causing violations, such as a particular ingredient exceeding an allergen constraint or a day exceeding a calorie limit. Server applies predetermined substitution rules to replace problematic ingredients with allowed alternatives that maintain, as closely as possible, the original nutritional profile and recipe structure. If substitution rules cannot resolve the violation, server constructs an updated prompt sentence that explicitly describes the detected problem and required corrections, and re-invokes the generative AI model to regenerate only the affected sections. As output, server produces a revised structured meal plan and shopping list that satisfy user constraints.

### Step 11:

Server selects optimal sales facilities for each shopping list item using cost and quality indices.

Server receives, as input, the constraint-compliant structured shopping list and the item-level cost and quality indices computed in Step 5. Server iterates through each item in the shopping list and compares indices across available sales facilities. Server applies an optimization algorithm, such as minimizing cost subject to a minimum quality threshold or maximizing a weighted sum of negative cost and quality score, to select a preferred sales facility per item. Server annotates each shopping list entry with the selected facility and updates cost summaries for the entire shopping trip or per facility. As output, server produces an optimized shopping list that includes item-facility assignments and estimated total cost and quality metrics.

### Step 12:

Server transmits the structured meal plan and optimized shopping list to terminal for user display.

Server receives, as input, a request from terminal to retrieve the latest meal plan and shopping list. Server queries the database to obtain the structured records generated in previous steps and organizes them into response objects categorized by day, meal type, and facility. Server serializes these objects into a format suitable for network transmission, such as a structured text or a data-interchange format, and sends the response over a secure connection to terminal. As output, server provides terminal with a complete, machine-readable representation of the meal plan and optimized shopping list.

### Step 13:

Terminal displays the meal plan and shopping list and captures user feedback.

Terminal receives, as input, the structured meal plan and shopping list transmitted by server.

Terminal renders the data on a graphical user interface, grouping meals by day and highlighting nutritional summaries and cost estimates. Terminal also displays the shopping list grouped by sales facility, with indicators for item quantities and estimated costs. Terminal allows user to perform actions such as approving or rejecting specific meals, editing ingredient quantities, changing selected sales facilities, and marking items as already in stock.

Terminal records these user actions as feedback events and composes a feedback message containing accepted items, rejected items, modifications, and comments. As output, terminal sends this feedback message to server.

### Step 14:

Server updates user preference information and refines future prompt sentences.

Server receives, as input, the feedback message generated by terminal in Step 13. Server analyzes the feedback to detect patterns, such as frequent rejection of certain ingredients or preference for particular cuisines or sales facilities. Server updates user preference records and statistical counters, associating changes with specific features such as ingredient identifiers, flavor profiles, and cost levels. Server then adjusts parameters used in planning target generation and prompt templates, such as increasing the weight of favored cuisines or decreasing the probability of proposing disliked ingredients. Server may precompute updated template fragments that explicitly mention new preferences. As output, server refines its internal models and prompt-construction rules so that subsequent prompt sentences and generated plans are more closely aligned with user behavior.

### Step 15:

Server arranges delivery of products or prepared food through an online transaction processing apparatus.

Server receives, as input, a user command issued from terminal to order some or all items from the optimized shopping list. Server selects the items marked for delivery, groups them by selected sales facility, and constructs order records including item identifiers, quantities, facility identifiers, delivery address, and payment information tokens. Server sends these order records to an online transaction processing apparatus via a defined network interface. Server receives confirmations or error messages from the transaction processing apparatus, updates order status records accordingly, and notifies terminal of the result. As output, server initiates and monitors the delivery process for selected products or prepared food based on the optimized shopping list.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional menu recommendation and shopping support systems typically rely on static rule sets, manually curated recipe databases, or simple filtering of user profiles. Such systems process heterogeneous data sources, including refrigerator inventory, user health metrics, store price information, and delivery options, in an ad hoc and siloed manner. As a result, they often fail to fully exploit available data, are unable to dynamically adapt to changing conditions, and place a heavy burden on the user to interpret recommendations and make cost-effective decisions.

From a computing technology standpoint, there are several problems. First, existing systems do not provide an integrated processing pipeline in which image recognition of stored food items, time-series health analysis, price and freshness normalization across multiple vendors, and external food provision options are jointly modeled as machine-interpretable constraints. Consequently, the processor cannot systematically transform raw multimodal data into a unified constraint set suitable as input to an advanced natural language generation engine. Second, known approaches that utilize generative models typically pass only coarse user descriptions or generic dietary tags as prompts. These prompts fail to encode structured computational results such as nutrition constraints, energy intake targets, budget constraints, vendor-specific cost-quality trade-offs, and historical user preferences. This leads to unstable or suboptimal outputs from the generative model, requires significant manual post-processing, and degrades system reliability and scalability.

Third, prior systems rarely establish a closed feedback loop between user interaction and the upstream computation of prompts. Selection histories and user satisfaction indicators are not consistently associated with the structured representation of generated menus and shopping lists. Without such association, the processor cannot programmatically update preference information and cannot refine the content of subsequent prompts in a data-driven manner. This limits the ability of the system to improve over time and increases computation waste due to repeated generation of unsuitable options.

Fourth, optimization of cost and quality across both retail purchases and external food provision is typically handled by simplistic heuristics or isolated modules. There is no unified computation in which a processor calculates integrated evaluation indices per item (combining unit cost and quality/freshness) and then compares these indices with external provision price and delivery conditions. Accordingly, the system cannot algorithmically choose a cost-effective mixture between home cooking and outsourced food provision, which results in redundant API calls, repeated user interactions, and inefficient use of processing resources.

In summary, there is a need for a computer-implemented system and method that: (i) unifies acquisition and normalization of multimodal data (inventory images, health time-series, multi-store price/freshness, and external menu/delivery conditions); (ii) computes structured constraint conditions and preference information; (iii) encodes these as a machine-generated prompt sentence for a generative AI model; (iv) parses and persists the generative output as structured data; and (v) closes the loop by updating preference information based on user feedback and selection history. Such a system would improve the technical operation of the underlying computing environment by enabling more efficient data processing, more stable and relevant generative outputs, reduced unnecessary user interaction, and optimized coordination between local computation and external service calls.

The specific processing by the specific processing unit 290 of the data processing device 12 in Application Example 1 is realized by the following means.

The present invention provides a server comprising a processor and a memory storing instructions that, when executed by the processor, cause the processor to perform operations comprising: receiving image data acquired by an imaging device that captures a storage space containing a subject; executing an object recognition process on the image data by using an image analysis learning model to generate inventory information including a type and a quantity of leftover food; acquiring biological measurement values and activity amounts from at least one portable terminal and at least one wearable terminal; generating user health state information based on the biological measurement values and the activity amounts and storing the user health state information as time-series data; acquiring product information from at least one external information providing apparatus via a communication network; normalizing price information and freshness indices for a plurality of sales locations based on the product information and storing the normalized price information and freshness indices; calculating evaluation values of cost and freshness for food materials corresponding to the leftover food; calculating constraint conditions including at least nutritional conditions, energy intake conditions, and budget conditions based on the inventory information, the user health state information, the price information, and the freshness indices; generating a prompt sentence that encodes the constraint conditions together with preference information of a user; transmitting the prompt sentence as input data to a generative learning model configured to perform natural language generation; receiving, from the generative learning model, a response sentence including a plurality of menu proposals satisfying the constraint conditions and the preference information and a shopping list including purchase candidate items corresponding to the respective menu proposals; parsing the response sentence to extract the menu proposals and the shopping list as structured data and generating display data for presentation of the menu proposals and the shopping list on a user interface; collating the menu proposals with provision menus of at least one external food provision apparatus to specify at least one provision candidate; calculating a plurality of delivery options including a delivery route, a delivery time, and a delivery cost based on the provision candidate and the shopping list; arranging food delivery via at least one online order processing apparatus according to a selected delivery option; and storing, in association with the structured data, a selection history and satisfaction information of the user with respect to the menu proposals acquired via the user interface and updating the preference information included in the prompt sentence based on an accumulation result of the selection history and the satisfaction information, thereby adjusting contents of subsequent menu proposals and shopping lists. This enables the computing system to transform heterogeneous input data into a unified machine-interpretable constraint representation, to programmatically construct and refine prompt sentences for a generative AI model, to obtain more stable and context-appropriate generative outputs with reduced post-processing, to optimize cost-quality trade-offs across retail and external food provision using explicit evaluation indices, and to implement a closed feedback loop that continuously adapts menu and shopping suggestions based on user behavior, thereby improving the overall efficiency, scalability, and technical performance of the server-based recommendation and ordering infrastructure.

The term “processor” refers to one or more hardware computing units, such as a central processing unit or a graphics processing unit, capable of executing instructions stored in a memory to perform data processing operations described herein.

The term “memory” refers to a non-transitory computer-readable storage medium, such as semiconductor memory, magnetic storage, or optical storage, that stores instructions and data for access by the processor.

The term “imaging device” refers to an electronic image acquisition apparatus, such as a digital camera or an image sensor integrated into a terminal, configured to capture image data representing a storage space containing at least one physical object.

The term “storage space” refers to any physical compartment or area used to store items, including but not limited to a refrigerator compartment, a pantry shelf, a cabinet, or a container interior, which can be captured by the imaging device.

The term “image data” refers to digital data representing visual information captured by the imaging device, such as pixel values encoded in formats including, but not limited to, JPEG or PNG.

The term “object recognition process” refers to a computational procedure in which the processor analyzes image data to detect, localize, classify, and optionally quantify objects present in the image data.

The term “image analysis learning model” refers to a machine learning model, such as a neural network or a deep learning model, that has been trained to perform image analysis tasks including object detection, recognition, or segmentation on input image data.

The term “inventory information” refers to structured data generated by the processor that represents at least a type and a quantity of physical items detected in the storage space.

The term “leftover food” refers to any food item or ingredient that remains available for consumption after prior use or meal preparation and is stored within the storage space.

The term “portable terminal” refers to a user-operated mobile electronic device, such as a smartphone or tablet, that can communicate with the server and provide data including biological measurement values and activity amounts.

The term “wearable terminal” refers to an electronic device adapted to be worn on a user's body, such as a wristband device or a smartwatch, and configured to measure or provide biological or activity-related data.

The term “biological measurement values” refers to numerical or categorical data representing physiological parameters of a user, including but not limited to body weight, heart rate, blood pressure, or other vital signs.

The term “activity amounts” refers to numerical data representing physical activity metrics of a user, such as step counts, calorie expenditure, exercise duration, or movement intensity.

The term “user health state information” refers to data derived from biological measurement values and activity amounts that characterizes a health condition or trend of a user over time.

The term “time-series data” refers to data points associated with respective timestamps, enabling the representation and analysis of changes in values, such as user health state information, over time.

The term “external information providing apparatus” refers to a remote computing system or service accessible via a communication network and configured to supply product information including price and freshness data.

The term “product information” refers to data describing goods offered by sales locations, including identifiers, names, prices, expiration dates, quality indicators, or other attributes.

The term “price information” refers to data representing a monetary cost associated with a product, including unit price or total price, optionally normalized to a standard quantity.

The term “freshness indices” refers to numerical or categorical indicators of the freshness or quality level of food products, derived from factors such as expiration dates, harvest dates, or quality ratings.

The term “sales locations” refers to retail outlets or distribution points, such as stores or warehouses, where products are offered for sale and for which separate price and freshness information can be obtained.

The term “evaluation values of cost and freshness” refers to computed values that represent an assessment of products or food materials by combining or aggregating cost-related information and freshness-related information.

The term “constraint conditions” refers to a set of machine-interpretable limitations or targets, including nutritional conditions, energy intake conditions, and budget conditions, that restrict or guide generation of menu proposals and shopping lists.

The term “nutritional conditions” refers to constraints related to nutrient composition of meals, such as required or maximum amounts of proteins, fats, carbohydrates, vitamins, or minerals.

The term “energy intake conditions” refers to constraints related to caloric intake of a meal or a set of meals, including target ranges or limits on total energy consumption.

The term “budget conditions” refers to constraints related to monetary expenditure for purchasing food items or meals, including maximum allowed costs or cost targets.

The term “preference information” refers to data representing user-specific likes, dislikes, tendencies, or historical behavioral patterns that influence selection or generation of menu proposals and shopping lists.

The term “prompt sentence” refers to a machine-generated or machine-formatted natural language or structured text that encodes constraint conditions, preference information, and other contextual data for input to a generative learning model.

The term “generative learning model” refers to a machine learning model, such as a generative AI model or a language model, configured to generate natural language text or structured content in response to an input including a prompt sentence.

The term “response sentence” refers to natural language or structured text output produced by the generative learning model in response to the prompt sentence, and including at least menu proposals and a shopping list.

The term “menu proposals” refers to recommended meal or dish configurations, each including at least a description of a dish and an associated set of required ingredients or items.

The term “shopping list” refers to a collection of purchase candidate items, including their names and optionally quantities, that are proposed to be acquired in order to realize corresponding menu proposals.

The term “purchase candidate items” refers to individual goods or ingredients identified in the shopping list as potential targets for purchase by the user.

The term “structured data” refers to data organized into a defined schema or format, such as key-value pairs, tables, or records, that facilitates programmatic access, storage, and processing.

The term “display data” refers to data formatted for presentation on a user interface, including text, layout information, or associated metadata required to visually present menu proposals and shopping lists.

The term “user interface” refers to a graphical, textual, or voice-based interface through which a user can view information generated by the system and provide input or selections.

The term “external food provision apparatus” refers to a remote computing system associated with a food service provider, such as a restaurant or catering service, that offers prepared food or meal services and can receive and process provision requests.

The term “provision menus” refers to collections of food items or dishes that can be prepared and supplied by the external food provision apparatus.

The term “provision candidate” refers to at least one item or combination of items in the provision menus that corresponds to or satisfies at least part of a generated menu proposal.

The term “delivery options” refers to alternative configurations for delivering goods or prepared food, each including parameters such as a delivery route, an estimated delivery time, and a delivery cost.

The term “delivery route” refers to a path or sequence of locations used to transport items from a provider to a delivery destination.

The term “delivery time” refers to an estimated or actual time interval required to complete delivery from a provider to a destination.

The term “delivery cost” refers to a monetary charge associated with providing and delivering items or prepared food to the user.

The term “online order processing apparatus” refers to a remote computing system or service configured to receive, process, and manage electronic orders for products or food services via a communication network.

The term “selection history” refers to recorded information indicating which menu proposals or options a user has previously accepted, rejected, or otherwise interacted with.

The term “satisfaction information” refers to data representing user feedback or evaluation regarding menu proposals, meals, or shopping results, such as ratings, comments, or inferred satisfaction indicators.

The term “objective information” refers to data describing at least one user goal or intent, including but not limited to weight management, fatigue reduction, mental load reduction, or optimization of nutrient intake.

The term “evaluation index” refers to a computed numerical or categorical metric used to assess and compare purchase candidate items by integrating unit-amount cost and a quality evaluation value derived from freshness indices or related factors.

The term “purchase source candidate” refers to a specific sales location proposed as a potential source from which a particular purchase candidate item in the shopping list may be obtained.

The term “provision price information” refers to data indicating the monetary cost of menu items or dishes offered by an external food provision apparatus.

The term “delivery condition information” refers to data describing terms under which delivery is provided by an external food provision apparatus, including delivery areas, time slots, fees, and other constraints.

The term “cost-effective combination” refers to a selection of one or more items or services from among home cooking options and external food provision options that minimizes or optimizes a cost-related or value-related objective under given constraints.

The term “home cooking” refers to preparation of food by the user or in the user's environment using ingredients purchased according to the shopping list.

The term “external provision” refers to supply of prepared food or meals by the external food provision apparatus to the user via delivery or pickup.

In one embodiment, a server cooperates with at least one terminal and at least one wearable terminal to implement the claimed system. The server includes at least one processor, at least one memory storing executable instructions and data, and a network interface coupled to a communication network. The terminal includes a processor, a memory, a display, a camera serving as an imaging device, and a communication module. The wearable terminal includes a processor, sensors for biological measurements, a memory, and a communication module. The server, the terminal, and the wearable terminal are implemented using general-purpose computing hardware, such as a multi-core central processing unit, an optional graphics processing unit, semiconductor memory, and network interface controllers. In one concrete example, the server uses a multi-core x86 or ARM processor, a graphics accelerator compatible with a general-purpose GPU framework, a relational database management system such as a SQL-based database, and a web framework such as a typical server-side web framework.

The server stores in the memory a plurality of software modules. These modules include an image reception and preprocessing module, an image recognition module implemented using an image analysis learning model such as a convolutional neural network constructed with a machine learning framework (for example, an open-source deep learning framework), a health data management module, a product information management module, a constraint calculation module, a prompt generation module, a generative AI interaction module for communicating with a generative AI model, a response parsing module, a recommendation management module, and an order orchestration module. The server also stores configuration data defining nutritional rules, energy targets, and budget rules, and stores user-specific preference information and historical interaction logs.

The terminal executes an application that communicates with the server via an application programming interface over a secure network protocol. The terminal uses the camera hardware and an operating system camera library to capture image data of a storage space, such as an interior of a refrigerator or a pantry shelf. The terminal encodes the captured image data in a compressed image format and transmits the image data to the server together with metadata including a user identifier, a capture timestamp, and optionally a storage-space identifier. The terminal also presents user interfaces for displaying menu proposals and shopping lists, and for accepting user selections, ratings, and other feedback. The terminal may further acquire health-related data from an operating system health platform or directly from the wearable terminal, and may forward such data to the server.

The wearable terminal measures biological measurement values and activity amounts by using onboard sensors, such as a heart-rate sensor, an accelerometer, a gyroscope, and optionally a blood-pressure sensor. The wearable terminal transmits the measured data to the terminal or directly to the server using a short-range wireless protocol or a network interface. The server receives biological measurement values and activity amounts and converts them into user health state information. The server stores the user health state information as time-series data in a database, associating each data element with a timestamp and a user identifier, and may compute derived metrics such as daily calorie expenditure, moving-average heart rate, or weekly blood-pressure trends.

The server receives image data from the terminal and stores the image data in a storage subsystem, such as a file system or object storage service. The image recognition module of the server loads an image analysis learning model from the memory. In one embodiment, the image analysis learning model is a convolutional neural network having multiple convolutional layers, pooling layers, and fully connected layers, trained to perform object detection and classification. For example, the model architecture may be similar to a region-based detection network or a single-shot detection network, with a backbone feature extractor followed by detection heads that output bounding box coordinates and class probabilities.

The server preprocesses the image data by resizing each image to a fixed resolution, normalizing pixel intensities, and converting the image into a tensor representation suitable for input to the neural network. The server executes forward propagation through the convolutional neural network, which applies learned filter weights and non-linear activation functions to extract visual features and to predict, for each region of interest, a probability distribution over classes and a bounding box. The server applies post-processing such as non-maximum suppression to reduce redundant detections and outputs a set of recognized items with associated confidence scores and bounding boxes.

The server converts the recognized items into inventory information by mapping each recognized class to a generalized item category, such as “poultry,” “leafy vegetable,” or “dairy product,” and by estimating quantities based on bounding box size, calibration parameters, and optionally prior knowledge about typical package sizes. The server stores the resulting inventory information in a structured data format, such as records containing fields for item category, estimated quantity, unit, freshness estimate, and timestamp. By using a trained neural network and structured mapping, the server reduces manual input requirements and improves accuracy and speed of inventory acquisition relative to manual enumeration.

The server acquires product information from one or more external information providing apparatuses via a communication network, for example by invoking web application programming interfaces exposed by online product databases. The server receives product information including raw price values, units, store identifiers, product categories, and freshness-related attributes such as expiration dates. The product information management module normalizes the raw price values to a common basis, such as price per standardized unit quantity, and computes freshness indices by converting expiration dates into a numeric score that decreases as the expiration time approaches. The server stores normalized price information and freshness indices in a database, indexed by generalized item categories and sales locations, and periodically updates this information to maintain current values.

The server calculates evaluation values of cost and freshness by combining normalized price and freshness indices through a predetermined function, for example, a weighted sum or a multi-criteria scoring function in which freshness is merged with cost into a single evaluation index per item and per sales location. In one variation, the server defines an evaluation index as a linear combination of standardized cost and standardized freshness, with weights configurable according to user priorities or system settings. By precomputing evaluation indices for item categories across multiple stores, the server can perform later optimization decisions with reduced computational overhead and lower response latency.

The server further calculates constraint conditions including nutritional conditions, energy intake conditions, and budget conditions. The constraint calculation module may use a nutrition database that maps item categories to approximate macronutrient and micronutrient values per unit quantity. The server integrates the inventory information and user health state information to compute, for an upcoming meal, target intake ranges for caloric energy and nutrients. For example, the server may derive a target energy value based on a user's daily goal and current consumption, and may adjust macronutrient targets for specific objectives such as weight reduction or blood-pressure management. The server also determines a budget constraint based on user-supplied budget preferences or prior spending patterns.

The server maintains preference information describing the user's taste preferences, disliked ingredients, preferred cooking styles, portion size preferences, and similar factors. The server derives preference information from explicit user input and from historical selection and satisfaction data, using statistical analysis or a lightweight recommendation model to assign preference scores to item categories or dish attributes. The server combines the constraint conditions and preference information into a prompt sentence. The prompt generation module produces a natural-language text that encodes: (i) user health state and objectives; (ii) current inventory information; (iii) price and freshness conditions; (iv) nutritional and energy targets; (v) budget constraints; and (vi) user preferences.

In one example, the server generates the following prompt sentence:
“The user is currently on a diet and needs a low-calorie, high-protein dinner.

Current health data: weight 68.5 kilograms, slightly elevated blood pressure, and today's active calories 450 kilocalories.

Leftover ingredients in the refrigerator: 300 grams of chicken breast and 150 grams of broccoli.

Store data: brown rice and olive oil are available at low prices and are fresh at several stores. The target for this meal is about 500 kilocalories with at least 30 grams of protein and reduced salt.

Please generate three candidate dinner menus that use the leftover ingredients as much as possible, and provide a shopping list for any additional ingredients, including approximate quantities.”

In another example, the server generates a prompt sentence for a different health objective:
“User health status: the user is trying to lose weight and has slightly high blood pressure. Current leftover ingredients: chicken breast, broccoli, and a small amount of tofu in the refrigerator.

Budget for tonight's dinner is under 1,000 yen.

Please generate a healthy meal plan for tonight, considering low salt and low calorie intake, and create a shopping list with approximate quantities and the most cost-effective items.”

The server transmits the prompt sentence to a generative AI model. The generative AI model may be implemented as a large-scale language model trained on text data and fine-tuned on recipe and nutrition-related corpora. In one embodiment, the generative AI model is a transformer-based neural network having an encoder-decoder or decoder-only architecture with multiple self-attention layers, feed-forward sublayers, and positional encodings. The generative AI model is trained using a maximum-likelihood objective or a similar loss function, such as cross-entropy between predicted tokens and ground truth tokens, and its parameters are updated by stochastic gradient descent or a variant thereof.

The server sends the prompt sentence as an input sequence of tokens to the generative AI model via an application programming interface. The generative AI model processes the prompt sentence and outputs a response sentence comprising natural-language text that includes a plurality of menu proposals and an associated shopping list. Because the prompt sentence explicitly encodes machine-computed numerical constraints and normalized evaluation values, the generative AI model is guided to generate outputs that are internally consistent with the computational results of the server, which improves stability and reduces the need for manual adjustments compared to generic prompts that do not encode such constraints.

The server receives the response sentence from the generative AI model and invokes the response parsing module. This module converts the free-form text into structured data by applying pattern recognition and extraction algorithms, such as rule-based parsers, regular expressions tuned to typical recipe formats, or a secondary lightweight sequence-labeling model that tags ingredients, quantities, and dish names. The server maps ingredient names in the generated menus and shopping list back to generalized item categories and checks consistency with nutritional constraints by referencing the nutrition database. If necessary, the server may adjust quantities or flag infeasible menus for exclusion before presenting options to the user.

The server generates display data that arranges the menu proposals and items of the shopping list into a user-interface-appropriate structure. The terminal receives the display data and renders menu cards with titles, brief descriptions, approximate calories, protein content, and cost estimates, as well as a shopping list grouped by store or category. The terminal allows the user to select one of the menu proposals, to request recalculation, or to adjust constraints such as budget or cooking time.

When the user selects a menu proposal, the server consults a database of provision menus provided by one or more external food provision apparatuses, such as remote ordering platforms associated with food service providers. The server performs similarity matching between the structured representation of the selected menu proposal and available provision menus, using attributes such as main ingredient type, cooking method, nutritional profile, and flavor style. The server identifies one or more provision candidates that approximate or match the selected menu proposal. The server then combines the provision candidates with the shopping list and the precomputed evaluation indices for retail purchases, and calculates multiple delivery options. Each delivery option includes a selected provider, an estimated delivery route, an estimated delivery time, and a delivery cost, which the server obtains from external systems or by applying route-estimation algorithms.

To select a cost-effective combination of home cooking and external provision, the server compares the evaluation indices for purchasing ingredients and preparing dishes at home with provision price information and delivery condition information from external providers. The server executes an optimization algorithm, for example a dynamic programming or integer-linear-programming routine, that minimizes a cost function under constraints such as total budget, maximum acceptable delivery time, and required nutritional values. This algorithmic selection of a home-cooking/external-provision mix constitutes more than a mere automation of human decision-making; the server exploits precomputed evaluation indices, normalized pricing and freshness data, and route-aware delivery cost estimates to rapidly evaluate a large combination space that would be infeasible to explore manually in real time.

The server arranges food delivery by generating machine-readable order data and transmitting the order data to an online order processing apparatus. The server includes in the order data the menu items or prepared dishes to be provided, the delivery address, the preferred delivery time window, and payment-related information in a tokenized or reference form. The server monitors order status via callbacks or polling and updates the user interface on the terminal with confirmation and tracking information. By managing order orchestration at the server side, the system can batch requests, reuse cached store and menu information, and thus reduce network traffic and response latency.

The server logs, in association with the structured data representing menu proposals and shopping lists, the selection history and satisfaction information obtained from the user via the terminal. Satisfaction information may be collected as explicit numerical ratings, textual comments, or inferred from behavior such as repeat selection or early cancellation. The server analyzes the accumulated selection history and satisfaction information using a statistical model, such as a matrix-factorization model or a simple collaborative-filtering scheme, to adjust preference scores for item categories, flavors, and cooking methods. The server then updates the preference information stored in the user profile and adjusts subsequent prompt sentences to reflect the learned preferences. For example, if the user repeatedly rejects dishes with high salt content and low vegetable content, the server increases weights associated with low-salt and vegetable-rich attributes within the preference information, and explicitly encodes these tendencies in future prompt sentences.

This feedback loop provides a technical improvement in that the server no longer constructs prompts in a static manner but instead uses machine-interpretable preference updates derived from structured historical data. As a result, the generative AI model receives more accurate contextual input over time, which stabilizes the distribution of generated outputs, reduces the frequency of unsatisfactory menus, and lowers processing overhead caused by repeated regeneration. The system thus improves the efficiency and reliability of interactions between the server and the generative AI model.

The described architecture and data flow provide several technical advantages that go beyond automation of human tasks. By normalizing heterogeneous data (images, health time-series, multi-store prices, freshness, and menu databases) into structured representations and by precomputing cost-freshness evaluation indices, the server reduces repetitive per-request computation and network traffic. The combination of convolutional image recognition for inventory detection and transformer-based language generation conditioned on explicitly encoded numeric constraints reduces human input errors and shortens response times. The closed feedback mechanism improves predictive accuracy in menu generation and shopping list proposals, which decreases the number of user interactions and system calls required to reach an acceptable outcome.

Alternative embodiments may vary specific implementation details while remaining within the scope of the claims. In one variation, the server deploys the generative AI model locally rather than via an external service, for example by running an optimized transformer model on a graphics processing unit or a specialized accelerator. In another variation, the image analysis learning model uses a different architecture, such as a vision transformer, and is trained using data augmentation techniques including random cropping, horizontal flipping, and color jittering, and a loss function combining classification and localization errors. Training may be performed offline using a dataset of labeled refrigerator images and corresponding inventory annotations, and the trained weights stored in the server memory.

In yet another embodiment, the health data management module may incorporate additional sensor data, for example sleep metrics, stress indicators, or environmental measurements, which are aggregated and transformed into extended user health state information. The constraint calculation module may then adapt nutritional and energy targets based on these expanded inputs. In a further embodiment, the response parsing module may employ a sequence-to-sequence model trained specifically to convert free-form recipe text into a structured schema, thereby improving parsing accuracy and reducing manual rule configuration.

The terminal may be embodied as a smartphone, a tablet, a dedicated kitchen device with an integrated display and camera, or any other user-operated device capable of running a client application and communicating with the server. The wearable terminal may be embodied as a wrist-worn device, a ring, a belt, or another form factor capable of measuring biological signals and transmitting them to the server.

In all of these embodiments, the server, the terminal, and the wearable terminal operate cooperatively to implement the claimed system. The server executes structured, non-conventional processing sequences: neural-network-based image recognition for inventory generation; time-series health analysis; multi-store normalization of price and freshness; computation of multi-dimensional constraints and evaluation indices; construction of constraint-rich prompt sentences; parsing of generative responses into structured data; and iterative preference adjustment based on historical feedback. These technical features, and their interaction as described, improve the performance and operation of the computing environment itself in terms of processing speed, accuracy of recommendations, data management efficiency, and communication overhead.

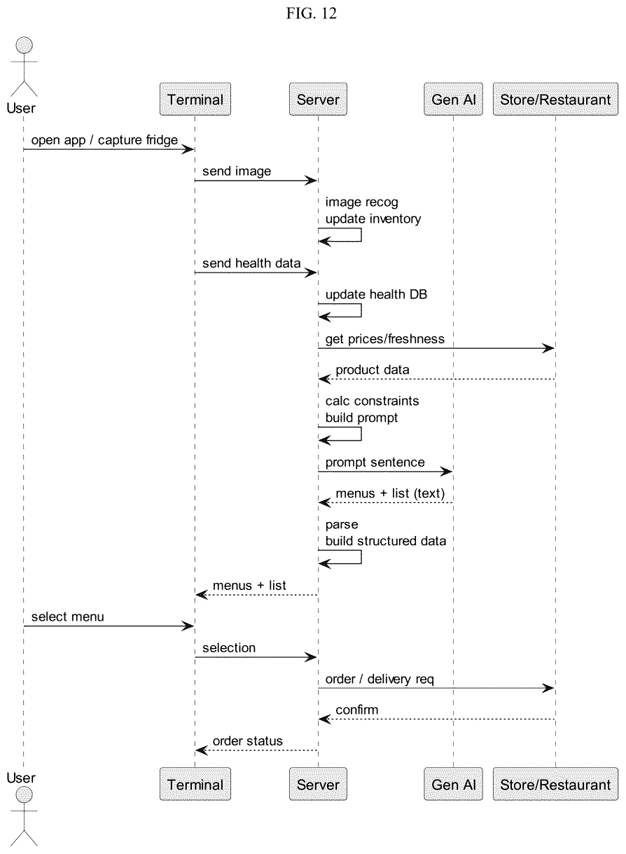

The following describes the processing flow using FIG. 12.

### Step 1:

The user operates the terminal to capture image data of a storage space.

The terminal activates a camera module, displays a preview on the screen, and, in response to a user operation, acquires one or more frames from the image sensor. As input, the terminal receives raw pixel data from the image sensor. The terminal encodes the raw pixel data into compressed image data (for example, JPEG) and attaches metadata including a user identifier and a timestamp. As output, the terminal generates a structured image-upload request containing the image data and the metadata and transmits this request to the server via a network interface.

### Step 2:

The server receives and stores the image data from the terminal.

As input, the server receives the image-upload request including the compressed image data and associated metadata. The server validates an authentication token and checks the integrity of the request. The server decodes the compressed image data into an internal image representation and writes the image file to a storage subsystem, while storing a reference to the file together with the user identifier and timestamp in a database. As output, the server produces a stored image record and a corresponding database entry that can be used as input to subsequent image recognition processing.

### Step 3:

The server performs image preprocessing for object recognition.

As input, the server obtains the stored image file corresponding to a particular user and time.

The server resizes the image to a fixed resolution, normalizes pixel intensities, and converts the image into a multidimensional tensor suitable for a convolutional neural network. The server may apply additional transformations such as color space conversion and mean-variance normalization. As output, the server generates a preprocessed tensor representing the storage space image, ready to be processed by an image analysis learning model.

### Step 4:

The server executes object recognition on the preprocessed image to generate inventory information.

As input, the server uses the preprocessed tensor and a set of learned weight parameters of a convolutional neural network model stored in memory. The server performs forward propagation through multiple convolutional layers, pooling layers, and fully connected layers to compute feature maps and classification scores. The server calculates bounding boxes and associated class probabilities, applies a non-maximum suppression algorithm to remove overlapping detections, and filters out low-confidence detections. The server then maps detected classes to generalized item categories and estimates quantities from bounding box sizes using calibration parameters. As output, the server produces structured inventory information, including for each detected item a category, an estimated quantity, a unit, and a confidence score, and stores this inventory information in the database linked to the user.

### Step 5:

The wearable terminal measures biological data and forwards health-related information.

As input, the wearable terminal continuously acquires sensor signals such as heart-rate waveforms, acceleration vectors, and optionally blood-pressure readings. The wearable terminal converts analog signals to digital values, aggregates the values over predefined time windows, and computes biological measurement values and activity amounts such as average heart rate, step counts, and calorie expenditure. The wearable terminal formats this data together with timestamps and a device identifier and transmits it to the terminal or directly to the server. As output, the wearable terminal provides structured health-related data packets that contain biological measurement values and activity amounts.

### Step 6:

The server receives and consolidates user health state information.

As input, the server receives health-related data packets from the terminal or the wearable terminal, including biological measurement values, activity amounts, timestamps, and a user identifier. The server validates the data range and consistency, and then writes each record into a time-series health data table in the database. The server may compute derived metrics such as daily total calories and moving averages of blood pressure by performing aggregation and smoothing operations over recent records. As output, the server generates updated user health state information that is stored as time-series data and can be retrieved and analyzed for constraint calculation.

### Step 7:

The server acquires and normalizes product information from external information providing apparatuses.

As input, the server initiates network requests to one or more external systems that supply product information, and receives responses containing raw price data, product identifiers, units, store identifiers, and expiration-related attributes. The server parses the responses, converts all price values to a normalized unit (for example, cost per 100 grams or per piece), and transforms expiration or harvest dates into numeric freshness indices using a predefined function that decreases as the expiration date approaches. As output, the server stores normalized price information and associated freshness indices for multiple sales locations in a structured product information table and maintains indexed access by generalized item category and store.

### Step 8:

The server calculates evaluation values of cost and freshness for potential purchase items.

As input, the server uses normalized price information and freshness indices for each item category across multiple sales locations. The server applies a cost-freshness scoring function, such as a weighted sum of normalized cost and normalized freshness, to compute a single evaluation value per item category and per store. The server may adjust weights according to user preferences or system configuration. As output, the server produces evaluation values of cost and freshness and stores them in an evaluation index table that can be rapidly accessed during shopping list and provider selection.

### Step 9:

The server computes constraint conditions based on inventory, health state, and product information.

As input, the server retrieves current inventory information for a user, recent user health state information, and normalized product information including prices, freshness indices, and evaluation values. The server references a nutrition database to map item categories to average nutrient contents and calculates current and target nutrient intakes, daily and per-meal energy targets, and recommended budgets based on the user's health objectives. The server then derives constraint conditions, including nutritional conditions, energy intake conditions, and budget conditions, by computing ranges or thresholds for nutrients and cost.

As output, the server generates a structured set of constraint conditions, represented for example as key-value pairs specifying target ranges and limits.

### Step 10:

The server updates and maintains user preference information.

As input, the server uses explicit user preferences (such as liked or disliked ingredients) and historical interaction data, including which menu proposals were accepted or rejected and corresponding satisfaction ratings. The server aggregates this data and applies a statistical model or heuristic scoring to assign preference scores to item categories, cooking methods, and flavor profiles. The server updates the user profile to reflect these scores and labels certain ingredients or styles as preferred or to be avoided. As output, the server maintains an up-to-date preference information structure that encodes user-specific tendencies for use in prompt generation.

### Step 11:

The server constructs a prompt sentence for a generative AI model.

As input, the server uses the structured constraint conditions, the current inventory information, the evaluation values of cost and freshness, and the updated preference information. The server combines these elements into a natural-language description that expresses user health status, leftover ingredients, nutritional and energy targets, budget limits, and taste preferences in a human-readable form. The server embeds specific numeric values (such as grams and kilocalories) and qualitative requirements (such as low salt or high protein) into a coherent text. As output, the server generates a prompt sentence tailored to the current context, for example:
“The user is currently on a diet and needs a low-calorie, high-protein dinner. Current health data: weight 68.5 kilograms, slightly elevated blood pressure, and today's active calories 450 kilocalories. Leftover ingredients in the refrigerator: 300 grams of chicken breast and 150 grams of broccoli. Store data: brown rice and olive oil are available at low prices and are fresh. The target for this meal is about 500 kilocalories with at least 30 grams of protein and reduced salt. Please generate three candidate dinner menus that use the leftover ingredients as much as possible, and provide a shopping list for any additional ingredients, including approximate quantities.”

### Step 12:

The server transmits the prompt sentence to a generative AI model and receives a response sentence.

As input, the server provides the generated prompt sentence and generation parameters such as maximum length and diversity settings to a generative AI model through a networked application programming interface or a local model inference interface. The generative AI model processes the prompt sentence and outputs a response sentence that contains natural-language descriptions of multiple menu proposals and an associated shopping list. The server waits for and receives this response sentence, which may be streamed or provided as a single text block. As output, the server obtains a text document that encodes candidate menus, ingredients, approximate quantities, and high-level nutritional and cost considerations.

### Step 13:

The server parses the response sentence to extract structured menu proposals and shopping lists.

As input, the server uses the received response sentence and a set of parsing rules or a secondary extraction model. The server tokenizes the text, identifies sentence boundaries, and applies pattern-matching or sequence-labeling techniques to detect dish names, ingredient names, and associated quantities. The server maps each detected ingredient to a generalized item category using a lookup table, checks for missing quantities, and fills gaps using default values if necessary. As output, the server creates structured data objects representing each menu proposal (including dish name, ingredient list, and estimated nutrition) and a consolidated shopping list (including purchase candidate items and required quantities).

### Step 14:

The server generates display data and sends proposals to the terminal.

As input, the server uses the structured menu proposals and shopping list data. The server formats this data into a response suitable for user interface rendering, including fields such as dish titles, brief textual descriptions, estimated calories, protein content, and a sorted list of items to purchase. The server encapsulates this data in a structured response message and transmits it to the terminal via the network interface. As output, the server produces display data that the terminal can directly render as menu cards and shopping lists.

### Step 15:

The terminal displays the menu proposals and shopping list to the user.

As input, the terminal receives the display data from the server. The terminal parses the structured content, populates user interface components such as lists, cards, and detail views, and presents the menu proposals and shopping list on the display. The terminal allows the user to scroll through the proposals, tap to view details, and select a preferred menu. As output, the terminal generates user interface events corresponding to user selections, modifications, or feedback.

### Step 16:

The user selects a menu proposal and optionally provides feedback.

As input, the user views the menu proposals and shopping list on the terminal screen and evaluates aspects such as taste, nutritional content, and cost. The user performs actions such as tapping a “select” control for a particular menu, entering a rating, or rejecting certain proposals. The terminal converts these actions into structured selection and feedback data. As output, the terminal transmits a selection message containing the selected menu identifier and optional satisfaction information back to the server.

### Step 17:

The server updates selection history and preference information based on user feedback.

As input, the server receives selection and satisfaction data from the terminal, including the identifiers of accepted or rejected menu proposals and any ratings or comments. The server stores these records in a selection history table linked to the corresponding structured menu and shopping list data. The server re-computes preference scores by aggregating new feedback with existing history, updating the user's preference information for item categories, flavors, and cooking styles. As output, the server maintains an updated selection history and preference profile that influences future prompt sentences and improves personalization.

### Step 18:

The server determines external provision candidates and calculates delivery options.

As input, the server uses the structured representation of the user-selected menu proposal, the normalized product information and evaluation values, and provision menus obtained from external food provision apparatuses. The server performs similarity matching between the selected menu and available provision options, identifies provision candidates, and retrieves corresponding provision price information and delivery condition information. The server then calculates multiple delivery options by combining provision candidates with the shopping list and by evaluating delivery routes, estimated delivery times, and delivery costs.

As output, the server produces a set of delivery options and a recommended cost-effective combination of home cooking and external provision based on computed evaluation indices.

### Step 19:

The server arranges food delivery via an online order processing apparatus.

As input, the server uses the selected delivery option, including provider identifiers, items or dishes to be delivered, address information, and desired delivery time. The server constructs an order payload according to the interface specification of the online order processing apparatus and transmits the payload over the network. The server receives order confirmation data such as an order identifier and estimated delivery time and updates corresponding records in its database. As output, the server generates order status data that can be reported to the terminal for user tracking of the delivery.

### Step 20:

The terminal presents order status and completion information to the user.

As input, the terminal receives order status updates from the server, including confirmation, dispatch, and completion notifications. The terminal updates on-screen indicators, such as order progress bars or expected arrival times, and may generate notifications when the delivery is imminent or completed. As output, the terminal provides the user with real-time feedback about the state of the ordered food and acts as the final presentation point in the end-to-end processing flow.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computerized meal-planning and shopping-assistance systems typically treat menu generation and shopping list generation as separate, rule-based operations. Such systems generally rely on static recipe databases, fixed filtering rules for dietary restrictions, and simple price lookups for products. As a result, these systems suffer from several technical limitations.

First, existing systems do not efficiently utilize a generative AI model in a way that is tightly integrated with structured user constraints such as dietary restrictions, user preferences, budget limits, and store-specific availability. The lack of a structured interface between user condition data and natural-language prompts leads to ad hoc prompt construction, inconsistent model behavior, and high variance in output quality. This degrades the reliability and predictability of the computer-generated results and increases computational waste due to repeated trial-and-error calls to the model.

Second, conventional systems do not provide an efficient mechanism on the server side to transform unstructured model output into a normalized, aggregated ingredient representation that is usable for downstream optimization. For example, slight variations in ingredient naming in the generated output often result in redundant or fragmented entries, which then require manual correction or additional processing. This leads to increased processing time, inefficient memory usage, and reduced scalability of server resources when handling many users in parallel.

Third, existing systems lack an integrated, server-centric workflow to map generated ingredient data to store- or location-dependent product information in an automated and computationally efficient manner. They do not optimize across purchasing locations or dynamically adjust menu content in response to budget overruns in a closed computational loop. Budget checks, substitutions, and store selection are often handled manually or via simplistic rules, resulting in suboptimal computational performance and frequent re-computation of large portions of the plan.

Fourth, many systems do not exploit the capabilities of generative AI models to generate multiple candidate plans under different constraint patterns (for example, different planning periods, cooking loads, or nutritional balances) from a single, structured condition set. Instead, they force the user to re-enter or reformulate conditions repeatedly. This leads to redundant computation, repeated network calls, and inefficient use of server-side processing and bandwidth.

Accordingly, there is a need for an improved computer-implemented technique in which a server-side processor (i) systematically converts user condition information into structured data and corresponding prompt sentences, (ii) drives a generative AI model in a controlled, constraint-aware manner, (iii) normalizes and aggregates model outputs into a unified ingredient representation, (iv) automatically generates location-dependent shopping lists with budget verification and iterative correction, and (v) efficiently produces multiple optimized menu and shopping-list variants under different objective and constraint patterns. Such a technique should enhance the determinism, efficiency, and scalability of the overall computer system, reduce manual intervention, and reduce unnecessary recomputation and network traffic.

The specific processing by the specific processing unit 290 of the data processing device 12 in Example 2 is realized by the following means.

The present invention provides a server comprising a processor and a memory storing instructions that, when executed by the processor, cause the processor to acquire user condition information including a dietary restriction, a preference, a budget upper limit, and a preferred purchasing location via a user terminal and convert the user condition information into structured data; to generate, from the structured data, at least one prompt sentence in a natural language that encodes the dietary restriction, the preference, the budget upper limit, and additional constraints, and to convert the at least one prompt sentence into input data for a generative AI model; to execute the generative AI model on a computing platform to obtain model output including menu candidates and corresponding ingredient data; to normalize and aggregate the ingredient data into an aggregated ingredient list; to retrieve, from a storage of product information, product entries associated with one or more purchasing locations and map the aggregated ingredient list to location-dependent shopping lists with computed unit quantities, unit prices, and total estimated costs; to compare the total estimated costs with the budget upper limit to determine budget conformity; and, when the budget upper limit is not satisfied, to automatically generate and apply, in an iterative manner, correction prompt sentences and/or substitution rules to adjust the menu candidates and the shopping lists until budget-conforming menus and shopping lists are obtained, and to transmit the budget-conforming menus and the shopping lists to the user terminal for presentation. This enables the computer system to perform constraint-aware, location-dependent menu and shopping-list generation in a deterministic and resource-efficient manner, to reduce manual post-processing of model outputs, to minimize redundant computation and network calls through structured prompt orchestration and iterative correction, and to improve the overall performance, scalability, and reliability of server-side meal-planning and shopping-assistance operations.

The term “processor” refers to a hardware information processing unit, such as a central processing unit or a graphics processing unit, that executes instructions to perform arithmetic, logical, control, and input/output operations for implementing the functions of the system.

The term “memory” refers to a hardware storage element, such as a volatile or non-volatile storage device, that stores instructions and data to be accessed and processed by the processor.

The term “user terminal” refers to an electronic device operated by a user, such as a mobile communication device, a tablet device, or a computing device, that includes at least one input component and at least one output component and communicates with the server over a communication network.

The term “user input/output unit” refers to a hardware or software interface component of the user terminal, such as a touch screen, a display, a keyboard, a pointing device, or an audio interface, through which the user inputs data and receives information.

The term “condition information” refers to information indicating constraints or preferences of the user, including at least a dietary restriction, a preference, a budget upper limit, and a preferred purchasing location, and optionally including additional constraints such as health state information, life goal information, and behavioral constraint information.

The term “dietary restriction” refers to a limitation or exclusion condition on consumable items, such as avoidance of certain ingredients, nutrients, or food categories, that should be reflected in a generated menu.

The term “preference” refers to a tendency, liking, or desired characteristic specified by the user, such as a favored taste, cuisine type, cooking style, or ingredient type, that is to be taken into account when generating a menu.

The term “budget upper limit” refers to a maximum monetary amount specified by the user or the system for purchasing required items, and serves as an upper bound for an estimated total expenditure amount in generating a shopping plan.

The term “preferred purchasing location” refers to a purchasing entity, such as a retail store, an online marketplace, or another goods-providing facility, designated by the user or the system as a preferred source of items to be included in a shopping list.

The term “structured data” refers to condition information that has been converted into a machine-processable format, such as a key-value representation, a record, or a vector, in which data elements are explicitly labeled or indexed for computational processing.

The term “prompt sentence” refers to a sequence of symbols in a natural language that encodes at least part of the condition information and is provided as an instruction or query to a generative AI model in order to cause the model to generate output data.

The term “correction prompt sentence” refers to a prompt sentence that includes an instruction to modify or refine previously generated content, such as an instruction to reduce cost or change constraints, and that is supplied to the generative AI model to cause generation of adjusted output data.

The term “generative AI model” refers to a machine-implemented model, such as a neural network model, configured to generate output data, including at least menu information and ingredient information, in response to input data derived from a prompt sentence.

The term “input data for a generative AI model” refers to data, including tokenized or encoded representations of a prompt sentence or structured condition data, that is supplied to the generative AI model to initiate a generation process.

The term “model output” refers to data generated by the generative AI model in response to input data, including at least a plurality of menu candidates and ingredient information corresponding to the menu candidates.

The term “menu candidate” refers to a proposed set of consumable items, such as one or more dishes or meals associated with a time period or a time slot, generated by the generative AI model based on the condition information.

The term “ingredient information” refers to data indicating one or more components required to prepare a menu candidate, including at least an ingredient identifier and optionally an amount, a unit, or preparation attributes.

The term “aggregated ingredient list” refers to a collection of ingredient entries obtained by normalizing, integrating, and summing ingredient information across multiple menu candidates, such that identical or similar ingredients are represented as unified items with corresponding total required quantities.

The term “information storage unit” refers to a storage resource, such as a database or a file storage system, that stores product information, including entries associated with purchasing locations, and is accessible by the processor.

The term “product information” refers to data describing an item offered by a purchasing location, including at least a product identifier, a product name, a sales unit, a unit price, and an association with a purchasing location.

The term “sales product information” refers to product information selected or retrieved in association with an ingredient, indicating that the product can be used to satisfy at least part of a quantity required for that ingredient.

The term “location-dependent shopping list” refers to a list of product entries that is generated for one or more purchasing locations based on an aggregated ingredient list, and that specifies, for each entry, at least a sales product, a required number of units, and a cost associated with the purchasing location.

The term “sales unit” refers to a packaged or measurable unit in which a product is offered for sale, such as a weight unit, a volume unit, a count unit, or a container unit.

The term “unit price” refers to a monetary value per sales unit for a product, as recorded in product information for a purchasing location.

The term “required number of units” refers to a quantity of sales units of a product computed to satisfy a required quantity of an ingredient as indicated in the aggregated ingredient list.

The term “estimated total expenditure amount” refers to a computed monetary amount obtained by summing, across products included in a shopping list, products of unit prices and required numbers of units.

The term “budget conformity” refers to a relationship indicating whether the estimated total expenditure amount is within or outside of the budget upper limit specified in the condition information.

The term “high-cost component” refers to an ingredient, a product, or a menu element that contributes disproportionately to the estimated total expenditure amount relative to other components in a menu or a shopping list.

The term “low-cost alternative component” refers to an ingredient, a product, or a menu element that has a lower cost than a high-cost component and can be used as a substitute while maintaining at least a part of the functional or culinary role of the high-cost component.

The term “additional condition” refers to an element of condition information beyond the dietary restriction, the preference, the budget upper limit, and the preferred purchasing location, and may include at least health state information, life goal information, or behavioral constraint information.

The term “health state information” refers to data describing a physiological or medical state of the user, such as weight, age, metabolic condition, or disease-related constraints, which may influence menu generation.

The term “life goal information” refers to data indicating an objective or target of the user, such as weight reduction, muscle gain, or stress reduction, that may guide constraints or optimization criteria in menu generation.

The term “behavioral constraint information” refers to data describing limits on user behavior, such as available cooking time, frequency of shopping, or access to preparation equipment, that may restrict or shape generated menus and shopping lists.

The term “prompt sentence pattern” refers to a predefined or dynamically constructed template of a prompt sentence that encodes a particular combination of constraints, such as a planning period, a number of items, a cooking load, or a nutritional balance, for use with the generative AI model.

The term “planning period” refers to a duration, such as a number of days or weeks, for which menus and shopping lists are to be generated.

The term “cooking load” refers to an indication of a level of effort or complexity associated with preparing dishes, such as preparation time, number of steps, or equipment usage.

The term “nutritional balance” refers to a condition relating to distribution or adequacy of nutrients across menus, such as calories, proteins, fats, carbohydrates, vitamins, or minerals.

The term “purchasing location” refers to an entity that sells products, such as a retail outlet, an online store, or another point of sale, and that is associated with product information in the information storage unit.

The term “evaluation value” refers to a numerical or categorical score computed for a product or purchasing location based on at least one quality index and at least one price index, and used for selecting optimal products or locations.

The term “quality index” refers to data representing a measure of product quality, such as freshness, rating, or category grade, used in computing an evaluation value.

The term “price index” refers to data representing a measure of product price, such as absolute price, normalized price, or price-per-unit, used in computing an evaluation value.

The term “optimal cross-location shopping plan” refers to a plan that selects, from multiple purchasing locations, products satisfying ingredient requirements while optimizing according to evaluation values that incorporate quality and price.

The term “online transaction processing unit” refers to a component configured to perform communication and transaction control related to ordering or arranging products or services over a communication network.

The term “food delivery arrangement information” refers to data specifying products, quantities, delivery destinations, and delivery providers to be used for arranging the delivery of food items based on a shopping plan.

In one embodiment, a server cooperates with at least one terminal operated by a user to implement the claimed system. The server comprises a processor, a memory, a non-transitory storage device, and a network interface. The terminal comprises a processor, a memory, a display, one or more input devices such as a touch panel or keyboard, and a communication interface. The server and the terminal are connected via a communication network such as the Internet using standardized communication protocols.

The server stores, in the storage device, program modules including a communication module, a condition-acquisition module, a prompt-generation module, a generative AI model execution module, a menu and ingredient interpretation module, an ingredient aggregation module, a product-mapping module, a budget-evaluation module, a menu-adjustment module, a variant-generation module, and an output-generation module. The server loads these modules into memory and causes the processor to execute them.

The terminal executes an application, such as a web application rendered in a browser or a native application, that implements a user interface for acquiring condition information and for presenting menus and shopping lists. The terminal transmits user inputs to the server as structured messages and receives structured responses from the server for display.

The server acquires condition information by receiving data from the terminal. The terminal displays input fields on the display, and the user inputs a dietary restriction, preferences, a budget upper limit, a preferred purchasing location, and optionally additional conditions such as health state information, life goal information, and behavioral constraint information. The terminal converts these inputs into a structured representation, for example a set of key-value pairs stored in memory, and transmits the representation to the server through the communication interface using a request-response protocol.

The server parses the received condition information and normalizes it into structured data.

The server maps free-form dietary restriction strings to canonical codes, such as mapping multiple language variants of “vegan” into an internal diet code. The server converts the budget upper limit into a numeric internal representation in a fixed currency unit. The server tokenizes preference expressions into a standardized feature set, such as “spicy,” “low-carb,” or “quick-cook,” represented as a multi-hot vector. The server also stores additional conditions as structured attributes such as planning period, maximum cooking time per day, and target macronutrient ratios.

The server generates at least one prompt sentence on the basis of the structured data. The server uses a prompt-generation module that applies a deterministic template to construct a natural-language instruction that encodes the dietary restriction, preferences, budget upper limit, preferred purchasing location, and additional constraints. The server constructs, for example, a prompt sentence of the following form:
“Generate a 7-day meal plan for one adult. The user follows a vegan diet and prefers spicy dishes. The total estimated cost of all required ingredients must not exceed 5,000 yen. Use only ingredients that can be purchased at the preferred store. Limit the total daily cooking time to 45 minutes and maintain a balanced distribution of protein, carbohydrates, and fats. Provide daily breakfast, lunch, and dinner menus, and list ingredients with approximate quantities for each dish. Finally, summarize all ingredients into a consolidated shopping list.”

In another example, the server generates a prompt sentence that emphasizes cost reduction: “Create a seven-day vegan meal plan for one adult with a strict budget of 5,000 yen in total. Prioritize low-cost ingredients and simple recipes. Use only ingredients available at the specified store. The user likes spicy and Asian-style dishes. For each day, provide breakfast, lunch, and dinner, and list ingredients and approximate amounts. Then provide a consolidated shopping list optimized to minimize total cost.”

The server converts each prompt sentence into input data for a generative AI model. In one embodiment, the server uses a neural network-based generative AI model implemented as a sequence-to-sequence Transformer architecture stored on the storage device. The server loads the model parameters into memory and uses a tokenization library to transform the natural-language prompt sentence into token identifiers. The server then constructs an input tensor comprising token identifiers and positional encodings. The server places the tensor on a processing device such as a graphics processing unit to reduce overall latency and increase throughput when serving multiple users.

The generative AI model comprises multiple encoder and decoder layers including multi-head self-attention sublayers, feed-forward sublayers, layer-normalization sublayers, and residual connections. The server configures the model with hyperparameters such as a hidden dimension, a number of attention heads, and a number of layers. The server trains or pre-trains the model on a corpus of recipe texts, menu plans, and ingredient lists using supervised learning or instruction-tuning techniques. During training, the server computes a loss function such as a cross-entropy loss between predicted token sequences and target sequences, and updates model parameters using gradient-descent optimization, such as an adaptive optimization method. The server may also apply regularization techniques such as dropout and weight decay, and use data augmentation such as paraphrasing of instructions or random perturbation of ingredient order to make the model robust to variations in prompt sentences.

The server uses the generative AI model execution module to perform inference. The server supplies the input tensor to the generative AI model and executes a decoding algorithm such as beam search or nucleus sampling. The server sets parameters including maximum generation length, temperature, and sampling thresholds in order to balance diversity and determinism. The model outputs a sequence of tokens that the server converts back into text using the corresponding vocabulary.

The server interprets the generated text using the menu and ingredient interpretation module. The server applies parsing rules and pattern-matching techniques to segment the generated text into a set of menu candidates, each associated with one or more meals. The server detects markers such as “Day 1,” “Breakfast,” “Lunch,” and “Dinner” and associates dish names and ingredient lists with these markers. The server uses regular expressions and a domain-specific grammar to identify ingredients, quantities, and units in each text line. The server converts this information into an internal menu data structure stored in memory, with fields for day index, meal type, dish title, ingredient identifier, amount, and unit.

The server uses a controlled normalization process to map ingredient strings to canonical identifiers. The server uses a reference table of ingredient names stored in the storage device and applies string-matching algorithms, such as token-based similarity and character-level distance metrics, to associate variations like “firm tofu,” “tofu (firm),” and “extra-firm tofu” with a single canonical ingredient identifier. The server resolves ambiguous strings by considering nutritional category, typical usage, and context such as associated dishes. This reduces fragmentation of data and improves the accuracy of subsequent aggregation.

The server aggregates ingredient information across all menu candidates using the ingredient aggregation module. The server groups ingredients by canonical identifier and sums numeric quantities after converting them to a consistent unit. For example, the server converts grams and kilograms to a single base unit by lookup in a unit conversion table. The result is an aggregated ingredient list in which each entry includes an ingredient identifier, a total required quantity, and a base unit. The server stores this aggregated list in memory as a specific data structure optimized for subsequent database queries, such as an indexed array or a hash map keyed by ingredient identifier.

The server maps aggregated ingredients to product information using the product-mapping module. The server stores product information in an information storage unit such as a relational database. The product information includes, for each product, a product identifier, a canonical ingredient key, a sales unit, a unit size, a unit price, and an associated purchasing location. The server executes database queries that retrieve, for each ingredient identifier in the aggregated ingredient list, products whose canonical ingredient key matches the identifier and whose purchasing location matches the preferred purchasing location or a candidate location set.

The server determines, for each ingredient, a combination of products and required numbers of units that cover the total required quantity. The server uses numerical calculations implemented in the product-mapping module to divide the total required quantity by the product unit size and round up to the next integer number of units. The server then multiplies the required number of units by the unit price to compute a product cost. The server accumulates these product costs across all ingredients to compute an estimated total expenditure amount.

The server evaluates budget conformity using the budget-evaluation module. The server compares the estimated total expenditure amount with the budget upper limit. If the total is within the limit, the server marks the plan as budget-conforming and stores this result. If the total exceeds the limit, the server triggers the menu-adjustment module.

The server performs menu adjustment by using non-conventional, rule-based operations that are specifically configured to operate on model outputs and product mappings. The server identifies high-cost components by sorting ingredients or dishes in descending order of their contribution to the estimated total expenditure amount. The server maintains a substitution table that associates high-cost ingredients with low-cost alternatives that satisfy the same dietary restriction and, where possible, maintain similar taste or nutritional properties. For example, the server may substitute a premium nut with a non-premium nut, or a specialty grain with a more common grain. The server modifies the ingredient list for affected dishes by replacing the high-cost ingredients with corresponding low-cost alternatives and recalculates the aggregated ingredient list and shopping list.

In another embodiment, the server uses correction prompt sentences to re-invoke the generative AI model when budget is exceeded. The server constructs a new prompt sentence that includes feedback from the budget-evaluation module. For example, the server may generate:
“The previously generated weekly vegan meal plan exceeded the budget limit. Regenerate a 7-day vegan meal plan for one adult with a maximum total ingredient cost of 5,000 yen. Prioritize cheaper ingredients and avoid premium products such as imported fruits or specialty nuts. Maintain spicy flavor where possible and use only ingredients available at the specified store. Provide updated daily menus and an ingredient list with approximate quantities.”

The server supplies this correction prompt sentence to the generative AI model with adjusted generation parameters, such as a lower temperature for more deterministic output, and repeats the interpretation, normalization, aggregation, product mapping, and budget evaluation. This iterative loop continues until budget conformity is achieved or a predefined iteration limit is reached. Because the server reuses structured data and intermediate results, the system reduces redundant computation and minimizes network traffic between server and terminal.

The server generates multiple menu and shopping-list variants using the variant-generation module. The server builds different prompt sentence patterns that encode distinct constraint sets, such as a low-effort cooking pattern with maximum daily cooking time, a high-protein nutritional pattern, and a stress-reduction life-goal pattern. The server feeds each pattern to the generative AI model and stores the resulting menus and shopping lists with associated metadata. The server then enables the terminal to present these variants to the user, allowing selection based on different optimization criteria without requiring the user to manually re-enter conditions. As a result, the server improves computational efficiency by structuring model calls and by centralizing constraint encoding.

The terminal receives the menus and shopping lists from the server and displays them on the display unit. The terminal groups menu information by day and meal and lists dishes with descriptive names and associated ingredients. The terminal also displays the location-dependent shopping list in a categorized form, such as by product category or store section. The user can inspect and, in some embodiments, locally mark items as already available at home, and the terminal transmits updates to the server so that the server can remove corresponding entries from the aggregated ingredient list and recalculate costs. This bidirectional communication, combined with the server-side aggregation and mapping, reduces the amount of data exchanged and improves responsiveness.

This architecture and processing flow provide technical effects beyond mere automation of human planning. The server reduces memory usage and processing time by enforcing canonical ingredient identifiers and by aggregating ingredients before database access, which reduces the number of database queries and the size of intermediate data structures. The deterministic prompt templates improve the consistency of generative AI outputs, which reduces the need for repeated, exploratory model invocations and thereby lowers computation time and energy consumption. The rule-based budget correction routine operates directly on structured ingredients and products, which avoids full re-generation of menus in many cases and provides faster convergence to a budget-conforming solution.

The server improves the accuracy of cost estimation by systematically mapping generated ingredients to concrete products with defined unit sizes and prices, instead of relying on approximate, user-estimated prices. The combination of normalized ingredient representation and product-level mapping enables high-precision cost calculations and reduces error accumulation. The system also improves scheduling and load balancing in a multi-user environment, because the modular structure of prompt generation, model execution, aggregation, and mapping allows independent scaling of each module on cloud infrastructure, such as assigning the generative AI model to accelerators while keeping database operations on separate nodes.

The generative AI model in this system does not simply replicate human reasoning but applies mathematically defined transformation rules across high-dimensional representations. The model internally represents menu structures and ingredient relationships as learned vector embeddings, exploits attention mechanisms to relate constraints expressed in the prompt sentence to candidate dishes, and outputs sequences that encode meals and ingredients consistent with those constraints. The server uses explicit control over model hyperparameters, decoding algorithms, and iterative correction prompts to guide this process in a way that improves determinism and quality relative to ad hoc human prompting.

Alternative embodiments can employ different neural network architectures, such as encoder-only or decoder-only Transformer models, or hybrid models that combine a language model with a structured recommendation model. The server can also use different optimization algorithms, such as integer linear programming, on top of the aggregated ingredient list and product information to refine the selection of products and to minimize cost or maximize quality under additional constraints.

In all these embodiments, the cooperation of the server, the generative AI model, and the terminal, together with the specific data structures and algorithms for structured condition encoding, prompt sentence generation, ingredient normalization and aggregation, location-dependent product mapping, budget evaluation, and iterative correction, provides an improved computer-implemented technique. The system achieves higher processing speed, improved accuracy of generated menus and shopping lists, reduced communication load, and enhanced scalability compared to conventional rule-based or unstructured prompt-driven solutions.

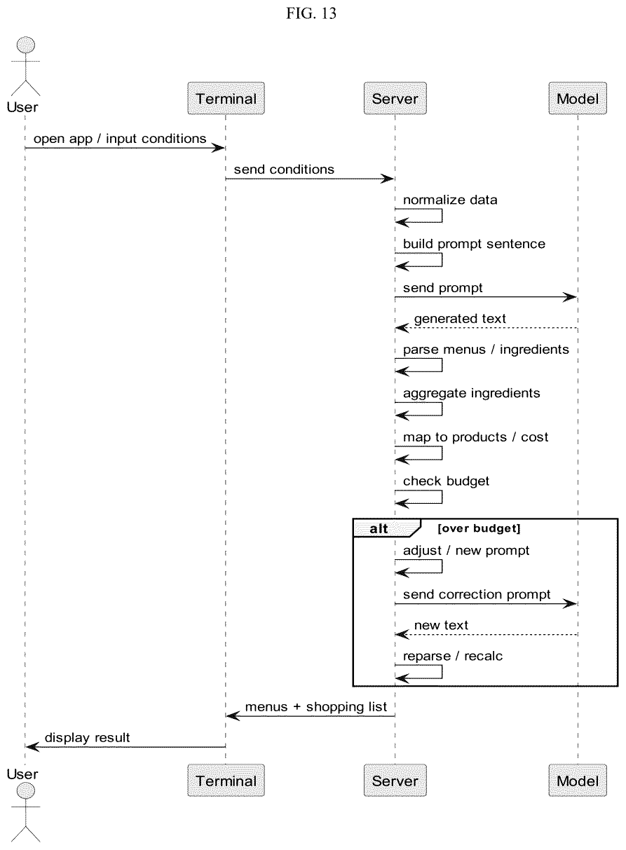

The following describes the processing flow using FIG. 13.

### Step 1:

The user operates the terminal to launch an application and open a condition-input screen.

The terminal displays input fields for a dietary restriction, preferences, a budget upper limit, a preferred purchasing location, and optional additional conditions such as health state, life goals, or behavioral constraints. The input of this step is the application start event; the output is a visible user interface ready to receive condition information.

### Step 2:

The user enters the condition information via the terminal. The terminal receives keystrokes, touch selections, and checkbox states and updates an internal data structure, for example a key-value map in memory, representing the current condition inputs. The input of this step is the user's raw interactions (touches, key presses, selections); the output is structured condition data held locally on the terminal.

### Step 3:

The terminal validates the condition data and transmits it to the server. The terminal checks simple rules, such as ensuring the budget upper limit is numeric and required fields are not empty. The terminal then serializes the condition data into a structured message, attaches any necessary authentication information, and sends the message via a network interface to the server. The input of this step is the internal condition data object created in Step 2; the output is a network request containing the normalized condition information delivered to the server.

### Step 4:

The server receives the condition information from the terminal and parses it. The server reads the incoming message from the network interface, decodes the structured format into a server-side data structure, and verifies that all required fields are present and of correct type. The input of this step is the received message containing the user's condition information; the output is a validated condition record stored in server memory.

### Step 5:

The server normalizes the validated condition record into internal codes and features. The server maps textual dietary restrictions to internal diet codes, converts the budget upper limit into a numeric value in a standard currency unit, and tokenizes preference strings into a set of standardized tags. The server also derives additional parameters such as a default planning period if none is specified. The input of this step is the validated condition record; the output is a normalized condition structure containing coded diet type, numeric budget, preference tags, and derived parameters.

### Step 6:

The server constructs a prompt sentence from the normalized condition structure. The server applies a deterministic template and inserts the coded dietary restriction, preferences, budget, preferred purchasing location, and any additional constraints into the template to form a natural-language instruction. For example, the server may generate a prompt sentence such as:
“Generate a 7-day meal plan for one adult. The user follows a vegan diet and prefers spicy dishes. The total estimated cost of all required ingredients must not exceed 5,000 yen. Use only ingredients that can be purchased at the preferred store. Limit the total daily cooking time to 45 minutes and maintain a balanced distribution of protein, carbohydrates, and fats. Provide daily breakfast, lunch, and dinner menus, and list ingredients with approximate quantities for each dish. Finally, summarize all ingredients into a consolidated shopping list.”

The input of this step is the normalized condition structure; the output is at least one prompt sentence in natural language.

### Step 7:

The server converts the prompt sentence into input data for a generative AI model. The server applies a tokenizer to the prompt sentence, generating token identifiers and positional encodings, and then organizes these into an input tensor suitable for the model. The server may also add special tokens indicating the beginning and end of the sequence. The input of this step is the natural-language prompt sentence produced in Step 6; the output is a model input tensor stored in server memory and optionally placed on a dedicated processing device.

### Step 8:

The server executes the generative AI model using the model input tensor. The server feeds the tensor to the generative AI model, such as a trained Transformer-based language model, and runs a decoding procedure (for example, beam search or nucleus sampling) with predetermined parameters like maximum length and temperature. The model performs numerical matrix operations and attention calculations to generate an output token sequence. The input of this step is the model input tensor; the output is a generated token sequence representing the model output.

### Step 9:

The server decodes the generated token sequence into model output text. The server converts token identifiers back into text using the model's vocabulary mapping and joins tokens into coherent sentences. The server may also clean up formatting markers or special tokens. The input of this step is the token sequence produced by the generative AI model; the output is a textual model output containing menu descriptions and ingredient lists.

### Step 10:

The server interprets the model output text to extract menu candidates and ingredient information. The server scans the text to detect day and meal markers (such as “Day 1,” “Breakfast,” “Lunch,” “Dinner”), segments the text accordingly, and identifies dish names and ingredient lines using pattern-matching and grammar rules. The server creates internal menu objects with fields for day index, meal type, dish name, and raw ingredient strings. The input of this step is the textual model output; the output is a structured set of menu candidates with associated ingredient entries stored in server memory.

### Step 11:

The server normalizes the ingredient entries into canonical ingredient identifiers. The server reads each raw ingredient string, compares it against a reference list of known ingredients, and uses similarity calculations to find a canonical ingredient key. The server replaces varied ingredient spellings or synonyms with the canonical key and converts textual quantities and units into numeric values and standardized units using a unit conversion table. The input of this step is the structured menu set with raw ingredient entries; the output is a normalized menu set where each ingredient is represented by a canonical identifier, a numeric quantity, and a base unit.

### Step 12:

The server aggregates ingredient quantities across all menu candidates. The server groups ingredient entries by their canonical identifiers and sums their numeric quantities after ensuring they use the same base unit. The server forms an aggregated ingredient list in which each item includes the canonical ingredient identifier and a total required quantity. The input of this step is the normalized menu set; the output is an aggregated ingredient list representing total ingredient requirements for the planned period.

### Step 13:

The server maps the aggregated ingredient list to product information at one or more purchasing locations. The server queries an information storage unit that holds product information, selecting products whose ingredient keys correspond to the canonical ingredient identifiers and whose purchasing location matches the preferred purchasing location or a candidate location. The server calculates, for each ingredient, the required number of units by dividing the total required quantity by a product's unit size and rounding up. The server then multiplies the required number of units by the unit price to determine the cost per ingredient. The input of this step is the aggregated ingredient list and the product information database; the output is a location-dependent shopping list containing product identifiers, required units, and per-ingredient costs.

### Step 14:

The server calculates an estimated total expenditure amount and evaluates budget conformity. The server sums the costs of all products in the location-dependent shopping list to compute the total estimated cost, then compares this total with the budget upper limit included in the normalized condition structure. The server sets a budget conformity flag indicating whether the total cost is within or exceeds the budget. The input of this step is the location-dependent shopping list and the budget upper limit; the output is the estimated total expenditure amount and a budget conformity determination.

### Step 15:

The server adjusts menus and the shopping list when the budget is not satisfied. The server identifies high-cost components by ranking ingredients or dishes according to their cost contribution and consults a substitution table to find lower-cost alternatives. The server replaces selected ingredients with alternatives in affected dishes and recalculates the aggregated ingredient list and shopping list. In another mode, the server constructs a correction prompt sentence that incorporates budget feedback, such as:
“The previously generated weekly meal plan exceeded the budget limit. Regenerate a 7-day meal plan for one adult with a maximum total ingredient cost of 5,000 yen. Prioritize cheaper ingredients and avoid premium products. Maintain the specified dietary restriction and preferences, and use only ingredients available at the specified store. Provide updated daily menus and an ingredient list with approximate quantities.”

The server feeds this correction prompt sentence back into the generative AI model by repeating Steps 7 to 14. The input of this step is the budget conformity determination and the current menu and shopping list; the output is an adjusted set of menu candidates and a revised shopping list that approach or satisfy the budget constraint.

### Step 16:

The server finalizes a budget-conforming menu and shopping list. The server selects the latest version of the menu and shopping list that satisfies the budget condition or that best approximates it under predefined rules. The server packages these data into a response structure that includes days, meals, dishes, ingredients, product details, and the total estimated cost. The input of this step is the adjusted menu and shopping list and the budget conformity flag; the output is a finalized response dataset ready for transmission to the terminal.

### Step 17:

The server transmits the finalized response dataset to the terminal. The server encodes the menu and shopping list data in a structured format and sends it through the network interface back to the requesting terminal. The input of this step is the finalized response dataset; the output is a network response message carrying the generated menus and shopping list.

### Step 18:

The terminal receives the response message from the server and parses the contained data.

The terminal converts the structured response into internal objects suitable for display, organizing menus by day and meal and grouping shopping list items by category or store section. The input of this step is the network response from the server; the output is terminal-side data structures representing the menu plan and shopping list.

### Step 19:

The terminal displays the menus and the location-dependent shopping list to the user. The terminal renders the menu plan on the display, for example in a calendar or list format, and shows the shopping list along with product names, quantities, and estimated total cost. The terminal may allow the user to scroll, expand item details, and mark items as already available. The input of this step is the terminal-side data structures from Step 18; the output is a graphical presentation on the display and, optionally, updated user interaction states.

### Step 20:

The user reviews the presented menus and shopping list and may request modifications. The user interacts with the terminal to adjust preferences, modify the budget, or change the preferred purchasing location, or to remove or add particular dishes. The terminal captures these interactions and updates its internal condition data. The input of this step is the displayed information and the user's decisions; the output is updated condition information reflecting the user's requested changes, which can then be sent again to the server by repeating the processing flow starting from Step 3.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented meal recommendation and shopping support systems typically perform rule-based filtering or simple scoring on static user profiles and item catalogs. Such systems often treat inventory data, health data, emotional state data, and store price data as independent inputs and do not unify them into a coherent, machine-interpretable context. As a result, generation of meal plans and shopping lists remains coarse-grained, and the system behavior does not adapt well to temporal changes in user behavior, purchasing tendencies, or emotional conditions.

Further, in many existing systems that use machine learning or recommendation techniques, the role of content generation models is limited to ranking or selecting pre-existing items. These systems do not dynamically construct task-specific natural-language instructions to a generative AI model, and therefore cannot fully exploit the expressive power of such models. Without a disciplined mechanism to transform heterogeneous sensor inputs, historical behavior data, and emotion estimates into a structured prompt sentence, the generative AI model cannot consistently output results aligned with strict constraints such as budget limits, nutritional needs, or real-time emotional states. This leads to unstable quality, inconsistent constraint satisfaction, and additional manual corrections by the user.

In addition, existing architectures generally treat external ordering systems and store databases as separate modules that are loosely coupled with recommendation logic. They do not integrate generative AI output with multi-store price, quality, inventory, and location information in a closed data-processing loop. Consequently, a meal plan generated by an AI model may not be efficiently realizable in the real world at reasonable cost, and may require the user to manually reconcile recipes with actual product availability and delivery options. Moreover, conventional systems do not systematically feed back user modifications and feedback into the process that constructs the prompts to generative AI models. Without continuous adaptation at the prompt-construction layer, the system cannot improve its internal representation of user behavior tendencies and purchasing tendencies. This limits the technical performance of the system in terms of relevance, constraint adherence, and computational efficiency, because the generative AI model is repeatedly queried with suboptimal, underspecified, or inconsistent instructions.

Therefore, there is a need for an improved computer-implemented system and processing method that: (i) unifies food stock information, biological information, product attribute information, emotional state information, and historical behavior and preference information into a structured context; (ii) algorithmically analyzes this context with machine learning techniques to estimate behavior tendencies and purchasing tendencies; (iii) automatically constructs, logs, and iteratively refines prompt sentences for a generative AI model in accordance with multiple objectives and constraints; and (iv) post-processes the generative AI output to generate executable shopping plans, optimal product combinations across multiple sales locations, and integrated food delivery orders. Such a system should enhance the operation of the computer itself by providing a specialized dataflow and control logic that reduce user intervention, improve the consistency and efficiency of AI model invocation, and yield technically improved outputs in terms of constraint satisfaction and end-to-end automation.

The specific processing by the specific processing unit 290 of the data processing device 12 in Application Example 2 is realized by the following means.

The present invention provides a server comprising a processor, a memory, and communication circuitry, the processor being configured to acquire, via sensor interfaces, food stock information from at least one inventory sensing apparatus; acquire, via a wearable interface, biological information of a user from at least one portable biological information acquisition apparatus; acquire, via a network interface, product attribute information for a plurality of sales locations from at least one external information source; estimate an emotional state of the user by processing image data from an imaging device and audio data from a sound acquisition device; retrieve, from the memory, history information including behavior history information and preference information associated with the user; analyze the history information by one or more machine learning algorithms to estimate a behavior tendency and a purchasing tendency of the user; construct, based on the estimated behavior tendency and purchasing tendency and on the acquired food stock information, biological information, product attribute information, and emotional state, at least one prompt sentence that explicitly encodes constraints and objectives and that instructs a generative AI model to generate at least one meal plan candidate and at least one purchase candidate; transmit the at least one prompt sentence to the generative AI model and receive text data generated by the generative AI model; parse the generated text data to extract meal configuration information and purchase target information; associate the purchase target information with the product attribute information to generate a shopping list including purchased items, quantities, and costs; evaluate candidate product combinations across the plurality of sales locations using the product attribute information to optimize, according to predefined criteria, at least one of total cost, movement burden, and quality index; extract, based on the meal configuration information and the shopping list, meal service candidates at a plurality of provision locations and select proposal candidates that satisfy budget conditions, nutrition conditions, and the emotional state of the user; provide the proposal candidates and the shopping list to a user interface for presentation to the user; update the meal configuration information and the shopping list in response to modification instructions from the user and, when a change in constraint or objective is detected, reconstruct and retransmit a refined prompt sentence to the generative AI model; and generate and transmit order information corresponding to a selected proposal candidate or the shopping list to at least one external order processing apparatus to arrange food delivery. This enables the computer system to transform heterogeneous sensor inputs and historical behavior data into dynamically optimized prompt sentences for a generative AI model, to obtain AI-generated meal and purchase proposals that are automatically reconciled with multi-store product, price, and availability data, and to close the loop by generating executable, constraint-compliant shopping plans and delivery orders with reduced user input and improved technical performance.

The term “food stock information” refers to data representing types, quantities, and freshness or expiration status of food items currently available in at least one storage location such as a refrigerator, pantry, or freezer.

The term “sensor” refers to a hardware component or assembly configured to detect a physical or chemical state associated with a food item or storage environment and to output corresponding electrical signals, including, for example, imaging sensors, weight sensors, identification tag readers, or environmental sensors.

The term “portable biological information acquisition apparatus” refers to an electronic device configured to be worn or carried by a user and to measure biological signals of the user, such as heart rate, activity level, or sleep pattern, and to transmit corresponding biological information to an external system.

The term “biological information” refers to data representing a physiological condition of a user, including but not limited to heart rate, activity amount, sleep duration, estimated energy expenditure, and other health-related metrics derived from body measurements.

The term “external information source” refers to a computing resource accessible via a communication network and configured to provide product-related data, including but not limited to price data, quality data, freshness data, and inventory data for items offered at sales locations.

The term “product attribute information” refers to data describing characteristics of products available at sales locations, including at least one of item category, brand, quantity, unit price, quality level, freshness, inventory status, and associated location information.

The term “sales location” refers to a physical or virtual point of sale such as a retail store, grocery outlet, or online marketplace at which food products or related items can be purchased.

The term “imaging device” refers to an apparatus configured to acquire image data representing at least part of a user or environment, such as a camera integrated into a terminal, and to output such data for subsequent processing.

The term “sound acquisition device” refers to an apparatus configured to capture acoustic signals, such as a microphone, and to output corresponding audio data for analysis.

The term “emotional state” refers to a condition associated with the user's affective status at a given time, including, for example, stress, joy, sadness, fatigue, or calmness, represented as one or more categorical labels, scores, or intensities.

The term “history information” refers to accumulated data associated with a user over time, including at least behavior history information, preference information, purchase records, consumption records, and prior interaction logs with the system.

The term “behavior history information” refers to data representing past actions of a user related to food selection, shopping, or meal consumption, including time, location, store choice, product choice, and ordering frequency.

The term “preference information” refers to data representing explicit or inferred likes, dislikes, and priorities of a user with respect to foods, cuisines, ingredients, price ranges, nutritional profiles, or service types.

The term “machine learning algorithm” refers to a computational method that learns a model from training data to perform tasks such as classification, regression, clustering, or prediction, including but not limited to neural networks, decision trees, and probabilistic models.

The term “behavior tendency” refers to a predicted pattern of future or typical user behavior derived from history information, including likely shopping days, preferred stores, and commonly selected item categories.

The term “purchasing tendency” refers to a predicted pattern of user purchasing behavior derived from past purchases and related data, including preferred price ranges, frequently purchased items, and sensitivity to promotions.

The term “generative AI model” refers to a trained computational model configured to generate new data, such as natural language text, in response to input conditions or prompts, and implemented, for example, by a neural network trained on large-scale datasets.

The term “prompt sentence” refers to a textual instruction or set of textual instructions supplied to a generative AI model, the instruction encoding constraints, objectives, and contextual information that guide the generative AI model in producing output.

The term “meal plan candidate” refers to a proposed structure of one or more meals, including meal times, dish descriptions, and associated ingredients, generated or derived by the system for potential adoption by the user.

The term “purchase candidate” refers to a proposed set of one or more items to be acquired, including associated quantities and product categories, generated or derived by the system as suitable for realizing at least part of a meal plan.

The term “text data generated by the generative AI model” refers to a sequence of characters or tokens produced by the generative AI model in response to a prompt sentence, the sequence including description of meals, ingredients, and related instructions.

The term “meal configuration information” refers to structured data derived from generated text that specifies a composition of meals, including dish names, required ingredients, quantities, preparation notes, and assignment to particular times or days.

The term “purchase target information” refers to structured data derived from generated text that identifies ingredients or products intended to be purchased, including at least item names and required quantities.

The term “shopping list” refers to structured data representing a set of items to be obtained by a user, including at least product identifiers or descriptions, quantities, and optionally prices and store associations.

The term “provision location” refers to a facility or service at which prepared food can be provided to a user, such as a restaurant, cafeteria, or food delivery kitchen.

The term “meal service candidate” refers to a potential offering from a provision location, including menu items or set meals that partially or fully correspond to a generated meal configuration.

The term “proposal candidate” refers to a recommendation instance presented to a user, including at least one of a meal plan, shopping list, or meal service candidate selected as satisfying one or more constraints.

The term “budget condition” refers to a constraint specifying an allowable amount of expenditure, including but not limited to per-meal, per-day, or per-period monetary limits.

The term “nutrition condition” refers to a constraint or target related to nutritional composition of meals, such as total caloric intake, macronutrient balance, or reduction of specified nutrients.

The term “user interface” refers to hardware and software components configured to present information to a user and receive input from the user, including displays, touch screens, input controls, and associated application logic.

The term “modification instruction” refers to an input from the user indicating a requested change to at least one of a meal plan, shopping list, or proposal candidate, including addition, removal, or substitution of items or constraints.

The term “external order processing apparatus” refers to a computing system operated by a third-party service that receives structured order information, processes such information, and coordinates fulfillment, including food delivery or product shipment.

The term “order information” refers to data specifying a request for provision or delivery of goods or services, including product identifiers, quantities, delivery address, timing preferences, and payment-related identifiers.

The term “objective information” refers to data specifying one or more goals for the system's operation, including goals related to weight management, nutritional balance, reduction of mental burden, or enhancement of a target emotional state.

The term “constraint condition” refers to a rule or limit applied during generation or selection of plans and lists, including constraints on time period, total budget, ingredient usage, ingredients to be excluded, output formatting, and explanation granularity.

The term “prompt sentence pattern” refers to a reusable template or structured arrangement of textual elements for forming a prompt sentence, the template including placeholder portions for inserting specific constraint conditions and context.

The term “generation target period” refers to a time range to which a generated meal plan or shopping plan is intended to apply, such as a single meal, a day, or a week.

The term “upper limit of budget” refers to a maximum allowable monetary value for a set of purchases or meals over the generation target period.

The term “preferred use ingredients” refers to ingredients that are designated as desirable by the user or system, such as ingredients already in stock or regularly enjoyed by the user.

The term “excluded ingredients” refers to ingredients that are prohibited or undesirable in generated plans due to factors such as allergies, dietary restrictions, or user dislikes.

The term “output format” refers to a prescribed structure or style in which generated text or data is to be presented, including organization into sections, lists, or tables.

The term “explanation content” refers to descriptive text that clarifies reasons for a recommendation, such as nutritional benefits, budget implications, or emotional support effects.

The term “total cost” refers to the aggregate monetary amount associated with acquiring all items in a given shopping list or product combination.

The term “movement burden” refers to an evaluation metric reflecting effort or inconvenience associated with visiting or interacting with sales locations, including travel distance, travel time, or number of separate locations.

The term “quality index” refers to a metric representing an estimated quality level of products or services, based on factors such as freshness, user ratings, or supplier data.

The term “use history” refers to data describing past interactions between the user and specific products, stores, or services, including purchase frequency, satisfaction ratings, and repeated selections.

The term “purchase plan” refers to a structured representation of decisions about where, when, and which products are to be acquired, taking into account constraints and optimization criteria.

The term “shopping route” refers to an ordered sequence of visits or transactions at one or more sales locations, designed to implement a purchase plan efficiently.

The term “food delivery arrangement” refers to a process of scheduling and confirming provision of food to a user, using order information transmitted to the external order processing apparatus.

In one embodiment, a server, a terminal, and various sensing and communication devices cooperate to implement the claimed system. The server includes at least one processor, a memory, a non-transitory storage device, and communication circuitry. The terminal includes at least one processor, a display device, an input device such as a touch panel or microphone, and communication circuitry. The server executes an application program implemented, for example, using a general-purpose programming language such as Python and a web framework such as a web-application framework. The server also executes machine-learning libraries such as a numerical computation framework, a deep-learning framework, and a statistical-learning library, and a data-analysis library such as a tabular-data processing library. The terminal executes a native application or browser-based application implemented, for example, using a mobile application framework or platform-native user-interface toolkits.

The server stores, in the storage device, program modules including at least: a data acquisition module, a feature-extraction module, a behavior and purchasing tendency estimation module, a prompt-construction module, a generative-model communication module, a parsing and post-processing module, an optimization module for multi-store product selection, and an order-generation module. The memory stores, during execution, data structures including user profiles, food stock records, biological-information records, product-attribute records, behavior history records, preference records, emotion-state records, and logs of prompt sentences and outputs of the generative AI model.

The server receives food stock information from sensors located at or near storage containers such as refrigerators or pantries. In one embodiment, the sensor includes an imaging device configured to capture images of the interior of a storage container, and an identification tag reader such as an RFID reader or barcode scanner. The server stores raw image data in an image table and raw tag readings in an identifier table. The server executes an image-recognition process, for example using an image-processing library and a convolutional neural network model trained with supervised learning, to segment the image, detect object regions, and classify each region into an ingredient category. The server associates each detected region with a food item identifier and an estimated quantity based on size, pixel area, or detected packaging. The server merges this result with tag readings, if available, to refine the identification and expiration dates by matching tags to known product records. The server stores the resulting structured food stock information as records including item identifier, quantity, and expiration data.

The server obtains biological information from a portable biological-information acquisition apparatus worn by the user, such as a wrist-worn device. The terminal receives raw sensor data from the portable apparatus via a short-range wireless protocol and transmits pre-processed time-series values to the server. The server stores heart rate, step counts, sleep estimates, and other metrics in a time-series table indexed by user identifier and timestamp. The server aggregates this biological information using the data-analysis library, for example by computing daily averages, rolling-window variances, and derived features such as resting heart rate or sleep regularity. The server thus generates feature vectors that reflect short-term and long-term biological trends.

The server acquires product attribute information from external information sources, such as application programming interfaces provided by store or marketplace systems. The server issues network requests via the communication circuitry, parses received structured responses, and stores product identifiers, unit prices, quality indicators, inventory status, and store-location coordinates into a relational schema in the storage device. The server maintains indices over frequently queried fields such as product category, store identifier, and price to enable efficient search and optimization.

The server estimates an emotional state of the user by analyzing image data from an imaging device and audio data from a sound acquisition device, which may be integrated in the terminal. In one embodiment, the terminal transmits raw or compressed audiovisual data to the server. The server executes separate models for facial-expression recognition and voice-based emotion recognition. For facial expressions, the server uses a convolutional or convolutional-recurrent neural network that receives localized facial images and outputs emotion scores over a fixed set of emotion categories (for example, stress, joy, sadness, fatigue). For voice, the server extracts acoustic features such as Mel-frequency cepstral coefficients, pitch contour, and energy envelope, and applies a recurrent or transformer-based network to classify the emotional tone. The server further processes any free-text input from the user describing the current mood using a natural-language processing pipeline based on a language-processing library or an external natural-language analysis service to compute sentiment polarity and emotion labels. The server fuses these multimodal estimates into a unified emotion state vector using, for example, a weighted linear combination or a small neural network trained to minimize cross-entropy against labeled emotion data. The server stores the unified emotion state vector in the emotion-state table.

The server retains, as history information, behavior history information and preference information collected over time. The server stores purchase histories, restaurant orders, meal selections, and recipe views with timestamps, store identifiers, and price information. The server stores preference information such as explicit “like” or “dislike” flags, manual ratings, and inferred cuisine or ingredient preferences computed from behavior patterns. The server uses this accumulated history together with newly acquired data to build a feature space representing the user's behavior tendency and purchasing tendency.

The server executes the behavior and purchasing tendency estimation module by loading relevant history information and recent biological and emotion data from storage, constructing feature vectors with the data-analysis library, and applying one or more machine-learning algorithms. In one embodiment, the server uses a recurrent neural network or temporal convolutional network implemented by a deep-learning framework to model sequences of purchases and visits to sales locations. The input sequence includes, for each time step, encoded store type, total spending, item categories, and time metadata such as day-of-week. The network outputs probability distributions over future shopping days, likely item categories, and preferred stores. The server trains this network offline or periodically using a supervised-learning regime with a loss function such as cross-entropy for classification outputs and mean-squared error for regression outputs. The server updates model weights via gradient descent or a variant such as Adam optimization. This results in a predictive model that is more accurate and efficient than manually defined rules, because the model learns non-obvious temporal dependencies and user-specific patterns from large volumes of data.

The server constructs a prompt sentence for a generative AI model based on the estimated behavior tendency and purchasing tendency and on the acquired food stock information, biological information, product attribute information, and emotional state. The server transforms the various feature vectors and structured records into a compact context representation. The server then applies a prompt-construction algorithm that selects and fills a prompt sentence pattern from a library of patterns. Each pattern is a human-readable template that includes placeholders for objective information (such as weight reduction support, nutritional balance improvement, mental burden reduction, emotional state enhancement), constraint conditions (such as generation target period, upper limit of budget, preferred use ingredients, excluded ingredients, output format, and explanation content), and context values (such as remaining ingredients and current emotion labels).

In one example, when the user is vegan, has a daily budget of 2,000 units of currency, prefers Italian dishes, and reports mild fatigue while the server detects remaining ingredients such as tofu, tomatoes, and spinach, the server constructs the following prompt sentence:
“The user is vegan, has a budget of 2,000 yen for today, prefers Italian food, and is slightly tired. The remaining ingredients in the fridge are tofu, tomatoes, and spinach. The user is allergic to nuts. Please propose a full-day Italian-style vegan meal plan and a detailed shopping list. Use the remaining ingredients as much as possible and choose foods that help maintain stable energy levels.”

In another example, when the server runs a scheduled analysis based on past purchases, the server constructs:
“Using the user's past grocery purchase data from the last three months, generate this week's shopping list and propose two new recipes that primarily use the items the user buys most frequently, such as chicken, eggs, bread, and cheese. Please output a shopping list with quantities and two recipe descriptions.”

In a further example, when the user indicates high stress, the server constructs:
“The user is under high stress today. Using stress-reducing foods such as dark chocolate, bananas, omega-3 rich fish, and complex carbohydrates, propose a dinner menu and dessert that can help reduce stress. The user has a 2,500-yen budget and is not allergic to any foods. Please include a shopping list and a short explanation for each dish.”

By explicitly encoding constraints, objectives, and multi-source context into the prompt sentence, the server improves the determinism and constraint satisfaction of the generative process, thereby enhancing the technical behavior of the computer system compared with an unspecified or generic prompt.

The server transmits the constructed prompt sentence to a generative AI model, for example a transformer-based language model trained on large text corpora. In one embodiment, the server accesses the generative AI model as a remote service via an application programming interface. The server supplies the prompt text and parameters such as maximum output length, sampling temperature, and top-k or top-p thresholds to control diversity and determinism. The generative AI model, which internally consists of multiple layers of self-attention and feedforward sub-networks, computes token probabilities conditioned on the prompt and generates an output sequence that describes meal plans, ingredients, and shopping lists.

The server receives the generated text and stores it in association with the prompt and session identifiers. The server parses the generated text using rule-based parsing and, optionally, auxiliary linguistic analysis modules. The server identifies sections corresponding to individual meals (for example, breakfast, lunch, dinner), extracts dish names, and identifies ingredient lines and quantities. The server transforms each ingredient line into a structured record containing at least an ingredient descriptor, a quantity, and a unit. The server maps these ingredient descriptors to canonical ingredient identifiers using a mapping table and natural-language similarity functions. The resulting meal configuration information and purchase target information are stored in intermediate data structures.

The server then executes an optimization process that uses product attribute information from multiple sales locations. For each ingredient in the purchase target information, the server retrieves matching products from the product-attribute tables, taking into account equivalence classes and allowed substitutions. The server computes, for each candidate product, a cost contribution equal to unit price multiplied by required quantity, a quality index based on stored quality metrics and user ratings, and a movement-burden cost based on the physical distance or number of locations involved. The server formalizes the selection problem as an optimization problem, such as a constrained knapsack or minimum-cost flow, and solves it using a combinatorial optimization algorithm or a mixed-integer programming solver. The optimization criteria include minimizing total cost and movement burden while satisfying quality thresholds and user constraints. This algorithmic selection yields a shopping list with specific products from specific sales locations that is optimized in a way not achievable by simple rules or manual selection.

The server similarly processes the meal configuration information to identify provision locations, such as restaurants or food service providers, that can supply dishes matching or approximating the generated recipes. The server queries a database of menu items and tags such as dietary attributes and cuisine types. The server matches each generated dish to menu entries using text similarity measures and tag intersections. The server then scores candidate provision locations based on distance to the user, expected cost, user ratings, and compatibility with the user's emotional state and preferences. The server selects proposal candidates that meet budget conditions, nutrition conditions, and emotional-condition constraints.

The server transmits, via the communication circuitry, the resulting meal plans, optimized shopping list, and proposal candidates for external provision locations to the terminal. The terminal displays, on its display device, structured views of the recommendations, including meal-by-meal breakdowns, itemized ingredients with quantities and prices, and lists of restaurants or providers with summary metrics. The user reviews the information and can input modification instructions by adding, removing, or substituting ingredients, changing budget constraints, or selecting different objectives (for example, focusing on low-salt meals instead of stress reduction).

The terminal transmits these modification instructions to the server. The server updates the meal configuration information and shopping list according to the modification instructions. When the modifications change key constraints or objectives, the server reconstructs a new prompt sentence that reflects the updated state and transmits it to the generative AI model without rebuilding the entire context from scratch. This incremental prompt reconstruction reduces computational overhead and network load because the server can reuse cached behavioral and product-attribute analyses, leading to improved processing speed and reduced latency.

The server also generates order information when the user approves a plan. For a shopping-plan case, the server converts the optimized shopping list into one or more structured orders addressed to external order processing apparatuses associated with particular sales locations. For a restaurant-based case, the server converts selected menu items into a delivery order to a food-delivery processing apparatus. In both cases, the server formats the order information according to the external system's interface specification and transmits the order via the communication circuitry. The external order processing apparatus arranges actual delivery of food items or prepared meals, thereby linking the computational process to real-world device control and logistics operations.

This architecture produces several technical effects. By organizing heterogeneous data (sensor readings, biological signals, text, and audiovisual input) into efficient data structures and feature vectors, the server reduces redundant data access and enables more accurate and faster inference of user behavior tendencies and purchasing tendencies. By using machine-learning models with explicit training processes, loss functions, and optimization algorithms, the server improves prediction accuracy of future behavior over conventional heuristic-based systems, thereby reducing erroneous recommendations and unnecessary recomputation. By algorithmically constructing prompt sentences that embed precise constraints and objectives, the server reduces variability in the generative AI model output, leading to lower post-processing complexity and less need for repeated model calls.

Furthermore, the optimization of product combinations across multiple sales locations, done via explicit cost and movement-burden modeling, reduces the total number of network calls and database queries needed to assemble a feasible shopping list. Because the server can predict likely shopping patterns and restrict search to high-probability sales locations and product categories, the system avoids exhaustive search over all products and stores, thereby reducing computational complexity and improving response time. This behavior represents an improvement to computer operation and data management itself, rather than a mere automation of human decision-making.

The system can be implemented in various alternative forms. In one embodiment, the behavior and purchasing tendency estimation module may employ gradient-boosted decision trees instead of recurrent neural networks. In another embodiment, the emotion fusion may use a probabilistic graphical model instead of a neural-based fusion. In a further embodiment, the optimization module may simplify to a greedy algorithm for smaller product sets, trading off optimality for speed in resource-constrained environments. The generative AI model may run locally on a specialized accelerator in the server, or remotely in a cloud environment accessed via an application programming interface. The terminal may be a smartphone, a tablet, a smart display in a kitchen, or a vehicle-mounted console. The system may support both a fully automated mode, wherein periodic prompt generation and order suggestion occur without explicit user requests, and an interactive mode, wherein the user triggers prompt generation and may iteratively refine preferences.

In all of these embodiments, the server uses a defined sequence of data transformations, feature extractions, model inferences, prompt constructions, text parsings, and optimization steps, which are specifically tailored to constrain and enhance the operation of a generative AI model in coordination with multi-source data and external ordering systems. This configuration allows the computer system to achieve improved processing accuracy, reduced latency, and lower communication and computation overhead compared with conventional systems that do not tightly integrate behavior modeling, prompt construction, generative text processing, and multi-store optimization.

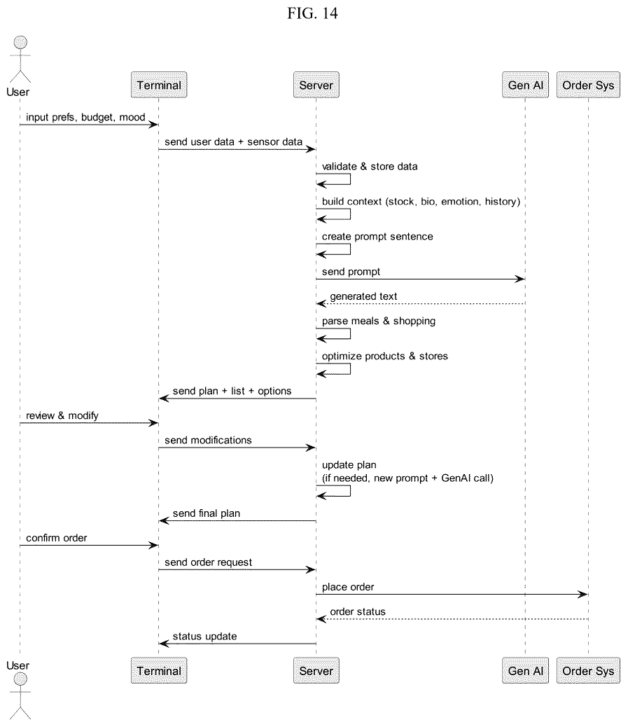

The following describes the processing flow using FIG. 14.

### Step 1:

The terminal acquires and transmits user and sensor inputs.

The user operates the terminal to input dietary restrictions, preferences, budget, and current mood through graphical forms or voice input. The terminal also acquires sensor data from a food-stock sensor, a portable biological-information apparatus, and built-in camera and microphone. Input to this step includes user-entered text (for example, “vegan, budget 2,000 yen, I feel a bit tired”), device readings such as heart rate and steps, food tag reads, and image/audio streams. The terminal converts these heterogeneous inputs into structured records, including JSON fields for diet type, allergies, budget, remaining items, biological metrics, and raw audiovisual data, and sends them via a network interface to the server. Output of this step is a structured request message containing user parameters and sensor data.

### Step 2:

The server validates, normalizes, and stores the received data.

The server receives the structured request message from the terminal and parses it into internal objects. Input to this step is the structured request message from Step 1. The server validates mandatory fields (for example, checks that budget is numeric and non-negative), and normalizes textual categories by mapping free-text diet labels and ingredient names to canonical codes stored in a reference table. The server detects and discards malformed or inconsistent entries, and may return an error to the terminal if critical fields are missing. The server then writes validated, normalized entries into relational tables for user sessions, food stock, biological information, emotion-related raw data, and preferences. Output of this step is a set of stored database records indexed by user identifier and session identifier.

### Step 3:

The server derives food stock information from sensor and image data.

The server reads raw image data and identification-tag data for the current session from storage. Input to this step includes stored images of the storage container interior and tag identifiers from the sensor. The server executes an image-recognition model, such as a convolutional neural network, to segment the image into object regions, classify each region into an ingredient category, and estimate relative size. The server then correlates these results with tag identifiers by matching spatial proximity or packaging patterns, and resolves conflicts by applying rules (for example, tag category overrides visual category when confidence is higher). The server converts this into structured food stock information with fields for item identifier, quantity estimate, and expiration date. Output of this step is an updated food stock table with structured records usable by later steps.

### Step 4:

The server aggregates and transforms biological information.

The server reads raw biological measurements from the biological-information table for the current and recent periods. Input to this step consists of time-stamped heart rate values, step counts, and sleep-duration estimates. The server groups these measurements into daily or hourly bins and computes summary statistics such as mean heart rate, maximum heart rate, total steps per day, and sleep-duration averages using a data-analysis library. The server may further derive features such as resting heart rate by averaging low-activity periods, and sleep regularity by computing the variance of sleep start times. These derived metrics are stored as feature vectors associated with the user and session. Output of this step is a set of biological feature vectors that characterize the user's current and recent physiological state.

### Step 5:

The server computes an emotion state from audiovisual and text data.

The server reads stored facial images, voice recordings, and free-text mood descriptions associated with the current session. Input to this step includes processed image frames of the user's face, audio segments of user speech, and text strings such as “I feel very stressed today.” The server applies a facial-expression classifier to each facial image to obtain emotion probability scores and applies a voice-based classifier to acoustic features extracted from the audio segments. The server applies a text-based sentiment/emotion analyzer to the free-text, generating sentiment polarity and discrete emotion labels. The server combines these three sources by computing a weighted average or feeding them into a small fusion network that outputs a unified emotion vector with components such as stress level, joy level, and sadness level. The server stores this vector as the current emotion state for the session. Output of this step is a unified emotion-state record representing the user's affective condition.

### Step 6:

The server updates behavior history and preference information.

The server accesses previous purchase records, menu selections, and ratings from storage.

Input to this step includes historical records of purchases at various sales locations, restaurant orders, previously generated meal plans, and explicit user feedback. The server appends the latest session's choices, if any, to this history, and updates preference scores for cuisines, ingredients, and price ranges by applying incremental statistics or a preference-learning model. For example, the server increases the preference weight for an ingredient that appears frequently in positively rated dishes. The server writes updated behavior history entries and preference weights back to their respective tables. Output of this step is an updated behavior history dataset and a set of preference scores that reflect the latest user interactions.

### Step 7:

The server estimates behavior tendency and purchasing tendency.

The server constructs temporal input sequences from the updated behavior history and preference scores. Input to this step includes sequences of store visits with timestamps, spending amounts, item categories, and preference weights. The server encodes each event into a feature vector and forms a time-ordered sequence for a fixed window, such as the last several weeks. The server feeds this sequence into a trained sequence model, such as a recurrent or temporal-convolutional neural network, and computes predicted probabilities over next-visit days, likely sales locations, and high-probability item categories. The server also estimates a purchasing tendency vector indicating sensitivity to price, frequency of bulk purchases, and responsiveness to promotions. The server stores these predictions as part of the session context. Output of this step is a behavior-tendency profile and a purchasing-tendency profile for the user.

### Step 8:

The server assembles a context representation for prompt construction.

The server collects, for the current session, the food stock information, biological feature vectors, emotion-state vector, updated preference scores, and behavior and purchasing tendencies. Input to this step is the set of structured outputs from Steps 3 through 7, combined with the user's current explicit inputs (diet type, allergies, budget, and objectives such as stress reduction). The server organizes this information into a context object, for example by allocating separate fields for constraints (budget, diet restrictions, time period), recommended focus (nutrition goals, emotional goals), and resource state (available ingredients, typical stores). The server then computes derived context features, such as “preferred primary store for this week” or “high-priority ingredient to consume before expiration.” Output of this step is a unified context representation that can be used to fill a prompt sentence pattern.

### Step 9:

The server constructs a prompt sentence for the generative AI model.

The server selects a prompt pattern from a library of predefined templates based on the objectives and context. Input to this step includes the unified context representation from Step 8 and an identification of the scenario type, such as daily meal planning, weekly shopping, or emotion-focused dinner suggestion. The server fills placeholders in the selected template with concrete values such as diet type, remaining ingredients, budget value, and emotion description. For a stress-reduction dinner case, the server may generate the prompt sentence: “The user is under high stress today. Using stress-reducing foods such as dark chocolate, bananas, omega-3 rich fish, and complex carbohydrates, propose a dinner menu and dessert that can help reduce stress. The user has a 2,500-yen budget and is not allergic to any foods. Please include a shopping list and a short explanation for each dish.”

The server verifies that all necessary constraints are present and logs the generated prompt sentence. Output of this step is an explicit prompt sentence text ready to be sent to the generative AI model.

### Step 10:

The server invokes the generative AI model and receives generated text.

The server sends the constructed prompt sentence to a generative AI model via an application programming interface. Input to this step is the prompt sentence and model control parameters such as maximum token count and sampling temperature. The server transmits these as part of a request message through the communication circuitry. The generative AI model processes the prompt with its internal neural architecture and returns a text sequence that describes meals, ingredients, and shopping recommendations. The server receives this response, extracts the generated text portion, and stores it along with a reference to the prompt and session. Output of this step is the raw generated text produced by the generative AI model in response to the prompt sentence.

### Step 11:

The server parses the generated text into structured meal and purchase data.

The server reads the generated text from storage. Input to this step is the raw text containing section headings (for example, “Breakfast”, “Lunch”, “Dinner”), recipe descriptions, lists of ingredients, and shopping lists. The server applies a parsing procedure that splits the text at known markers, identifies lines that describe ingredients, and extracts quantities and units using regular expressions and lexical rules. The server converts each ingredient line into a structured entry with fields for ingredient descriptor, quantity, unit, and associated meal. The server also identifies an overall shopping-list section and converts its lines into purchase target entries. The server saves meal configuration information and purchase target information into respective tables. Output of this step is a structured representation of meals and ingredients that can be matched with product data.

### Step 12:

The server maps ingredients to concrete products and optimizes multi-store selection.

The server takes the purchase target information and queries product-attribute tables for matching or substitutable items. Input to this step includes ingredient descriptors, required quantities, and constraints such as budget, preferred stores, and excluded ingredients. The server maps each descriptor to one or more canonical ingredient identifiers and fetches product records that satisfy dietary and quality constraints. The server then formulates an optimization problem in which variables represent choices of products at different sales locations, and objective terms represent total cost and movement burden, subject to constraints on quantity and budget. The server applies a suitable optimization algorithm to select a set of products that minimize cost and burden while maintaining quality thresholds. The server computes total estimated cost and attaches these product selections and costs to the shopping list. Output of this step is an optimized shopping list that specifies which products to buy at which sales locations, plus cost estimates.

### Step 13:

The server selects provision-location proposals based on meal configurations.

The server reads meal configuration information and accesses a database of menu offerings and attributes for provision locations such as restaurants. Input to this step includes dish descriptions and ingredient sets from the generated meal plan, as well as records of menu items tagged with cuisine type, dietary properties, and approximate ingredients. The server computes similarity scores between generated dishes and menu items using text similarity and tag matching, filters out incompatible items (for example, non-vegan items when the context requires vegan), and ranks candidate provision locations according to distance, price, rating, and emotional suitability. The server then selects a limited number of top-ranked proposal candidates for each meal. Output of this step is a list of proposal candidates: pairs of provision locations and menu items that correspond to or approximate the generated meals.

### Step 14:

The server sends recommendations to the terminal and the terminal displays them.

The server prepares a response message that contains the structured meal plan, optimized shopping list with product-level details, and proposal candidates with associated metrics. Input to this step is the structured result set from Steps 11 through 13. The server formats this information as a structured payload and transmits it to the terminal via the communication circuitry. The terminal receives the payload, parses it, and renders interactive views on the display, such as daily meal calendars, expandable recipe details, shopping lists grouped by store, and lists of recommended restaurants. Output of this step is the visual and interactive presentation that the user can see and manipulate on the terminal.

### Step 15:

The user reviews and modifies proposals and the terminal sends modification instructions.

The user examines the displayed meal plans, shopping list, and provision-location proposals on the terminal. Input to this step is the rendered content shown in Step 14. The user may perform actions such as deleting a dish, substituting an ingredient, increasing or decreasing budget, or choosing a different objective (for example, focusing on weight loss instead of stress reduction). The terminal captures these actions as modification instructions, which reference specific data elements (such as “remove ingredient X from meal Y” or “increase daily budget to 3,000 yen”). The terminal packages these instructions in a structured message and sends them to the server. Output of this step is a set of user-generated modification instructions transmitted to the server.

### Step 16:

The server updates plans according to modifications and, if needed, reconstructs a prompt sentence.

The server receives modification instructions from the terminal. Input to this step includes references to meals, ingredients, budgets, and objectives that the user wishes to change. The server applies these changes to the stored meal configuration information and shopping list by updating or deleting records and recalculating affected quantities and costs. If the modifications change critical constraints (such as diet type or primary objective), the server recognizes the change and executes the prompt-construction module again to generate a new or revised prompt sentence that reflects the updated context. The server may call the generative AI model again with this revised prompt to obtain an adjusted plan. Output of this step is an updated set of structured plans, potentially derived from a newly generated text, that satisfy the user's revised constraints.

### Step 17:

The user confirms the plan and the server generates order information.

The user selects a final plan to execute, such as accepting the optimized shopping list or choosing a particular provision-location proposal for delivery. Input to this step is a confirmation or selection operation on the terminal, which the terminal sends to the server as a selection message. The server interprets the selection and constructs order information that encodes specific products to buy or menu items to order, the desired quantities, delivery address, and timing preferences. For each external service involved, the server formats a dedicated order object compliant with that service's interface. The server then sends the order information to external order processing apparatuses via the communication circuitry and stores order identifiers and status in an orders table. Output of this step is a set of submitted orders in external systems and corresponding internal order records.

### Step 18:

The server monitors order status and the terminal presents delivery updates.

The server receives status notifications from external order processing apparatuses or periodically polls them. Input to this step includes status messages such as “accepted,” “preparing,” or “out for delivery,” along with estimated delivery times. The server updates the corresponding entries in the orders table and computes derived states such as remaining time until delivery. The server then pushes summarized status updates to the terminal. The terminal displays these updates to the user, including confirmation details and progress indicators. Output of this step is a real-time view of order status presented on the terminal, closing the loop from context acquisition to execution of a concrete shopping or delivery plan.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

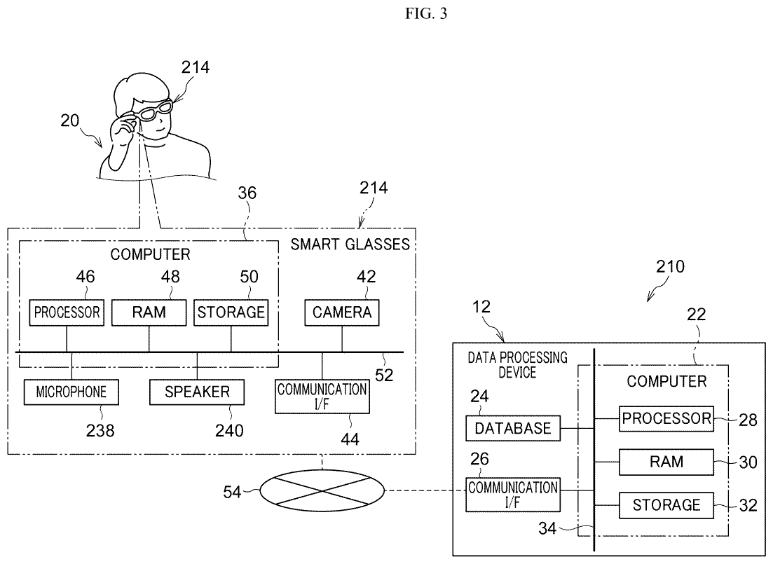

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

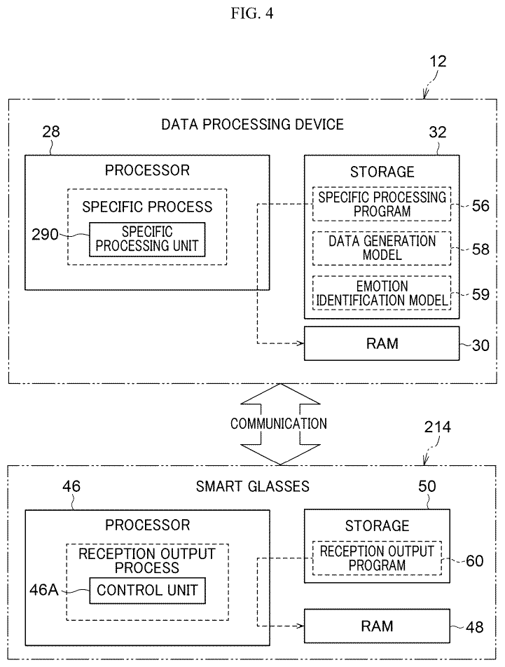

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

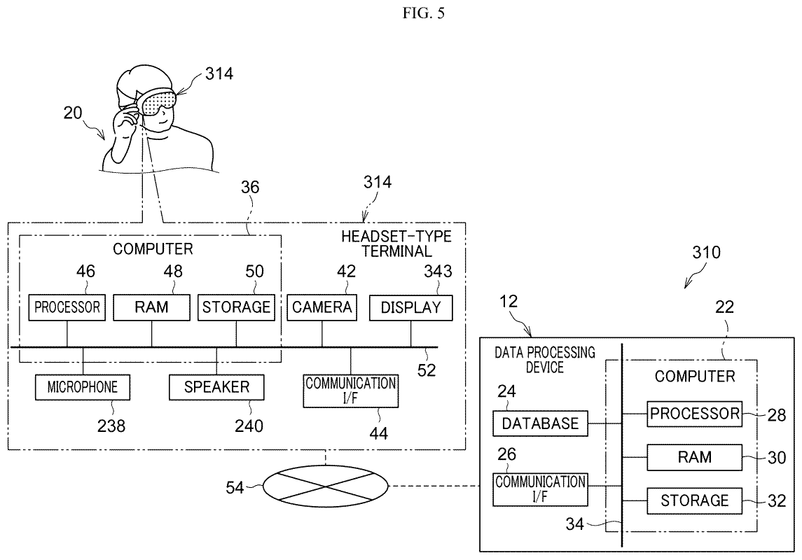

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

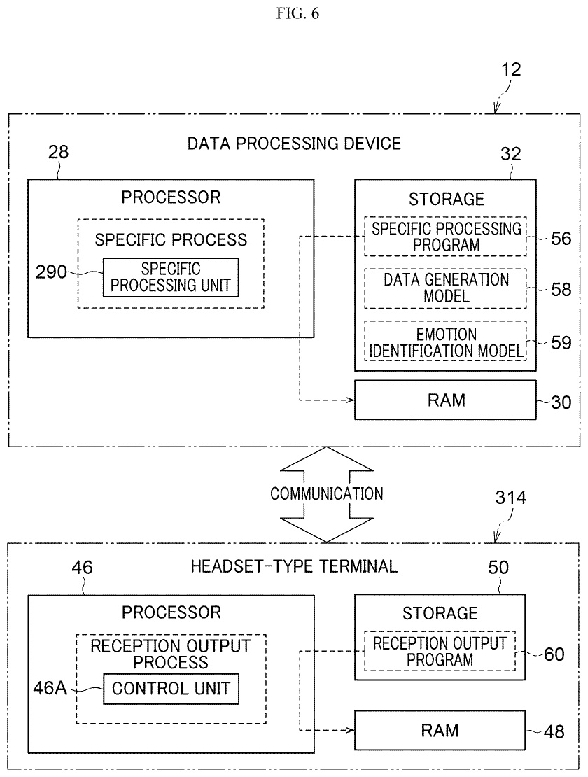

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

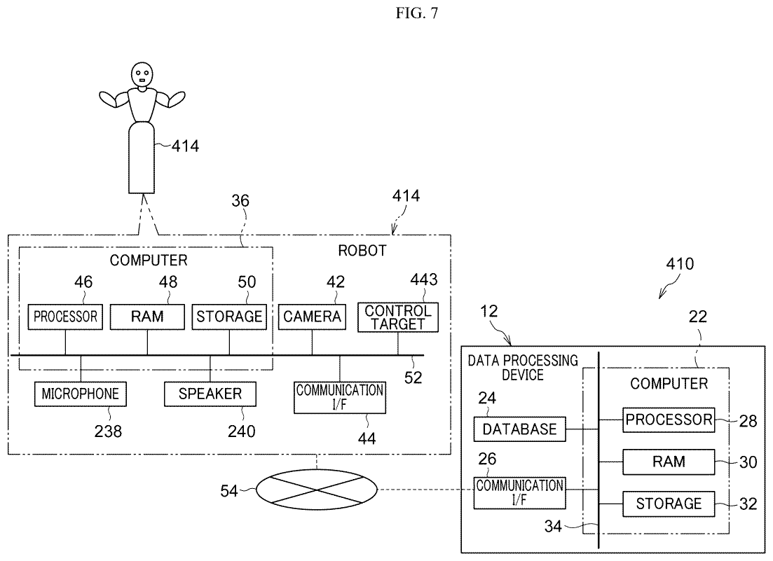

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

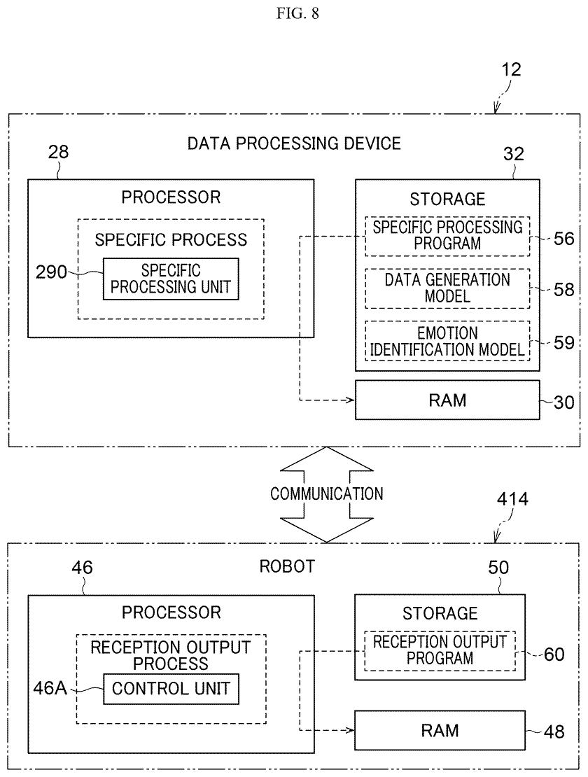

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

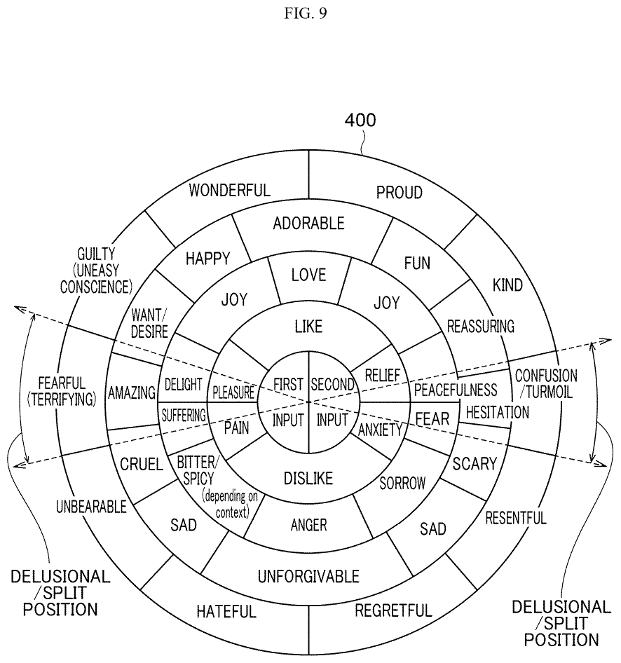

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

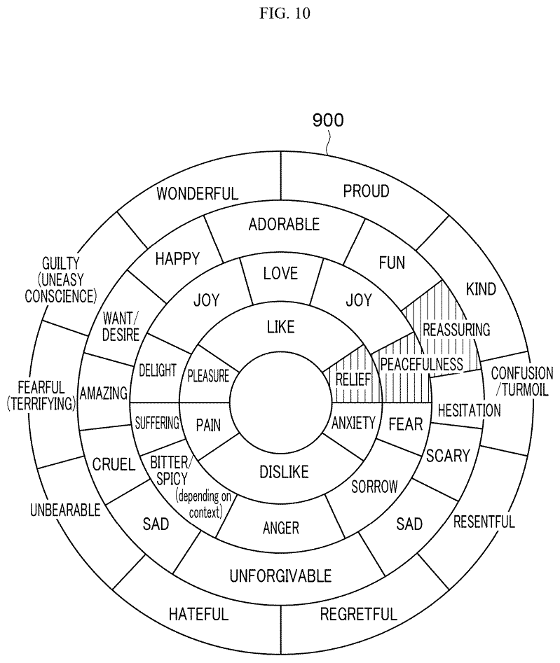

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system comprising a processor,
wherein the processor is configured to
acquire inventory information of food stored in a storage apparatus by using a sensor device and an imaging device,
acquire health information including physiological information and behavioral information of a user from a biological information acquisition device,
acquire price information and quality information relating to a plurality of sales facilities via a communication network,
generate planning target information including a nutritional goal and preference conditions of the user on the basis of the inventory information, the health information, the price information, and the quality information, and construct the planning target information as a natural-language prompt sentence,
input the prompt sentence to a generative AI model to cause the generative AI model to generate a meal plan for a predetermined period and a shopping list including purchase candidates for the plurality of sales facilities,
analyze the meal plan and the shopping list obtained from the generative AI model, convert the meal plan and the shopping list into structured information including items, quantities, nutritional values, and candidate sales facilities, and, when a component that violates a constraint condition of the user is detected, correct the meal plan and the shopping list by removing or replacing the component,
cause a user interface device to display the meal plan and the shopping list on the basis of the structured information, acquire preference information and modification operations from the user via the user interface device, and update the planning target information and the prompt sentence based on the preference information and the modification operations,
execute a regeneration process for the generative AI model by using the updated prompt sentence and iteratively improve the meal plan and the shopping list so as to reflect the preference information of the user, and
arrange delivery of products or prepared food via an online transaction processing apparatus on the basis of the shopping list and the price information and the quality information relating to the plurality of sales facilities.

### (Supplementary 2)

The system according to supplementary 1,
wherein the processor is configured to
include, in the prompt sentence, purpose information including at least one of weight reduction, nutritional balance improvement, mental load reduction, time saving, and cost reduction, and historical preference information of the user, and diversify candidate patterns of the meal plan and the shopping list generated by the generative AI model according to the purpose information.

### (Supplementary 3)

The system according to supplementary 1,
wherein the processor is configured to
calculate, on the basis of the price information and the quality information relating to the plurality of sales facilities, a cost index and a quality index for each item, select, for each item included in the meal plan and the shopping list, a sales facility that optimizes at least one of the cost index and the quality index, and include a result of the selection in the prompt sentence to be input to the generative AI model so as to propose a purchasing route with high cost effectiveness.

### Application Example 1

### (Supplementary 1)

A system comprising a processor,
wherein the processor is configured to
receive image data acquired by an imaging device that captures a storage space containing a subject, and execute an object recognition process on the image data by using an image analysis learning model, thereby generating inventory information including a type and a quantity of leftover food,
acquire biological measurement values and activity amounts from a portable terminal and a wearable information terminal, and generate user health state information based on the biological measurement values and the activity amounts, and record and manage the user health state information as time-series data,
acquire product information from an external information providing apparatus via a communication network, normalize price information and freshness indices for each of a plurality of sales locations based on the product information, store the normalized price information and freshness indices, and calculate evaluation values of cost and freshness for food materials corresponding to the leftover food,
calculate constraint conditions including nutritional conditions, energy intake conditions, and budget conditions, based on the inventory information, the user health state information, the price information, and the freshness indices, and generate a prompt sentence including the constraint conditions and preference information of a user,
transmit the prompt sentence as input data to a generative learning model that performs natural language generation, and acquire, from the generative learning model, a response sentence including a plurality of menu proposals satisfying the constraint conditions and the preference information, and a shopping list including purchase candidate items corresponding to the respective menu proposals,
analyze the response sentence, extract the menu proposals and the shopping list as structured data, and generate display data for presentation of the menu proposals and the shopping list on a user interface,
specify a provision candidate by collating the menu proposals with provision menus of an external food provision apparatus, calculate a plurality of delivery options including a delivery route, a delivery time, and a delivery cost based on the provision candidate and the shopping list, and arrange food delivery via an online order processing apparatus, and store, in association with the structured data, a selection history and satisfaction information of the user with respect to the menu proposals acquired via the user interface, and update the preference information included in the prompt sentence based on an accumulation result of the selection history and the satisfaction information, thereby adjusting contents of subsequent menu proposals and shopping lists.

### (Supplementary 2)

The system according to supplementary 1,
wherein the processor is configured to
extract objective information including at least one of weight management, fatigue reduction, mental load reduction, and nutrient intake optimization, from the user health state information and user input information, and generate the prompt sentence including the objective information, thereby causing the generative learning model to generate a plurality of objective-specific menu proposals and shopping lists.

### (Supplementary 3)

The system according to supplementary 1,
wherein the processor is configured to
calculate, for each purchase candidate item, an evaluation index obtained by integrating a unit-amount cost and a quality evaluation value based on the price information and the freshness indices of the plurality of sales locations, select a purchase source candidate for each purchase candidate item included in the shopping list based on the evaluation index, and compare provision price information and delivery condition information acquired from the external food provision apparatus with the evaluation index, thereby selecting a cost-effective combination between home cooking and external provision and arranging food delivery via the online order processing apparatus.

### Example 2

### (Supplementary 1)

A system comprising a processor,
wherein the processor is configured to
acquire condition information including a dietary restriction, a preference, a budget upper limit, and a preferred purchasing location of a user via a user input/output unit of a user terminal and generate the condition information as structured data,
generate, on the basis of the structured data, a prompt sentence in a natural language that expresses the dietary restriction, the preference, and the budget upper limit as conditions, and convert the prompt sentence into input data to be input to a generative AI model,
cause the generative AI model to operate on an information processing platform to generate, in response to the input data, model output including a plurality of menu candidates for a predetermined period and ingredient information corresponding to the menu candidates, aggregate the ingredient information included in the model output, normalize and integrate identical or similar ingredient names, calculate a required quantity for each ingredient, and generate an aggregated ingredient list,
acquire, from an information storage unit that stores product information associated with the preferred purchasing location, sales product information corresponding to the aggregated ingredient list, and generate a location-dependent shopping list by specifying, on the basis of the sales product information, a sales unit, a unit price, and a required number of units for each ingredient,
calculate an estimated total expenditure amount on the basis of the unit price and the required number of units for each sales product included in the shopping list, and determine budget conformity by comparing the estimated total expenditure amount with the budget upper limit, when the estimated total expenditure amount exceeds the budget upper limit, adjust the menu candidates and the shopping list by generating and re-inputting to the generative AI model a correction prompt sentence that instructs cost reduction, or by replacing high-cost components with low-cost alternative components, and generate a budget-conforming menu and a budget-conforming shopping list, and
transmit the budget-conforming menu and the location-dependent shopping list as output data to the user input/output unit of the user terminal and visually present the budget-conforming menu and the location-dependent shopping list via the user input/output unit.

### (Supplementary 2)

The system according to supplementary 1,
wherein the processor is configured to
generate the prompt sentence by adding, to the condition information, additional conditions including at least one of health state information, life goal information, and behavioral constraint information of the user, generate a plurality of types of prompt sentence patterns that define, in the natural language on the basis of the additional conditions, constraints relating to at least one of a planning period, a number of items, a cooking load, and a nutritional balance, and input the plurality of types of prompt sentence patterns to the generative AI model so as to cause the generative AI model to generate a plurality of menus and shopping lists corresponding to different purposes and different constraints.

### (Supplementary 3)

The system according to supplementary 1,
wherein the processor is configured to
generate the location-dependent shopping list by acquiring product information corresponding to a plurality of purchasing locations, extracting, for each purchasing location, sales product candidates with respect to the aggregated ingredient list, calculating evaluation values on the basis of a quality index and a price index, selecting a purchasing location and a sales product that satisfy a predetermined condition of the evaluation values to generate an optimal cross-location shopping plan, and outputting, via an online transaction processing unit, food delivery arrangement information for a plurality of delivery providers on the basis of the optimal cross-location shopping plan.

### Application Example 2

### (Supplementary 1)

A system comprising a processor,
wherein the processor is configured to
acquire food stock information by using a sensor,
acquire biological information of a user from a portable biological information acquisition apparatus,
acquire product attribute information regarding a plurality of sales locations from an external information source via a communication network,
estimate an emotional state of the user by using an imaging device and a sound acquisition device,
acquire, from a storage device, history information including behavior history information and preference information of the user based on the food stock information, the biological information, the product attribute information, and the emotional state, and analyze the history information by a machine learning algorithm to estimate a behavior tendency and a purchasing tendency of the user,
construct a prompt sentence for instructing a generative AI model to generate a meal plan candidate and a purchase candidate based on the estimated behavior tendency and purchasing tendency and on the food stock information, the biological information, the product attribute information, and the emotional state,
analyze text data output from the generative AI model to extract meal configuration information and purchase target information, and generate a shopping list including purchased items, quantities, and costs by associating the purchase target information with the product attribute information,
extract meal service candidates at a plurality of provision locations based on the meal configuration information and the shopping list, and select proposal candidates suitable for budget conditions, nutrition conditions, and the emotional state of the user,
present the proposal candidates and the shopping list to the user via a user display apparatus, update the meal configuration information and the shopping list in response to a modification instruction from the user, and, when necessary, reconstruct the prompt sentence for the generative AI model, and
generate and transmit order information to an external order processing apparatus based on a selected proposal candidate or the shopping list to arrange food delivery.

### (Supplementary 2)

The system according to supplementary 1,
wherein the processor is configured to
select or generate, as the prompt sentence, a plurality of types of prompt sentence patterns including constraint conditions such as a generation target period, an upper limit of budget, preferred use ingredients, excluded ingredients, output format, and explanation content, based on objective information including at least one of weight reduction support, nutritional balance improvement, mental burden reduction, and emotional state enhancement, and based on the behavior tendency of the user, and input the plurality of types of prompt sentence patterns to the generative AI model so as to comprehensively propose different meal plan candidates and shopping lists corresponding to respective objectives.

### (Supplementary 3)

The system according to supplementary 1,
wherein the processor is configured to
use the product attribute information acquisition and the shopping list generation to calculate, for each ingredient extracted from the generative AI model, combinations of candidate products by using price information, quality information, inventory information, and location information regarding the plurality of sales locations, evaluate the combinations of candidate products based on at least one of total cost, movement burden, quality index, and use history of the user, determine a purchase plan or a shopping route in which cost effectiveness and quality are optimized, and perform food delivery arrangement according to the determined purchase plan via the external order processing apparatus.

What is claimed is:

1. A system comprising:
circuitry configured to:
apply a convolutional neural network-based object recognition model to image data captured by an imaging device to generate object classification results and quantity estimates for items in a monitored storage location, and acquire time-series physiological data and behavioral data from a wearable biological sensor device via a communication interface coupled to a packet-switched network;
apply a trend detection algorithm to the time-series physiological data and behavioral data to generate a user state summary, and generate constraint condition data and preference condition data by combining the object classification results, the quantity estimates, the user state summary, and external condition data acquired from external data sources via the communication interface;
encode the constraint condition data and the preference condition data as a structured natural-language prompt sentence and input the prompt sentence to a generative AI model to generate an operational scheduling output and an acquisition output list for a predetermined period;
tokenize and parse the operational scheduling output and the acquisition output list into structured records comprising item identifiers, quantity values, numerical parameters, and source identifiers, apply a constraint violation detection algorithm to each structured record to identify violations of the constraint condition data, and remove or replace violating records; and
transmit the structured records to a terminal device via the communication interface, receive modification operations from the terminal device, update the constraint condition data and the prompt sentence based on the modification operations, and re-invoke the generative AI model using the updated prompt sentence to iteratively refine the operational scheduling output and the acquisition output list.

2. The system according to claim 1, wherein the circuitry is configured to
include in the prompt sentence purpose information comprising at least one of a constraint parameter type and an optimization objective, and historical preference information of the user, and diversify candidate patterns of the operational scheduling output and the acquisition output list generated by the generative AI model according to the purpose information.

3. The system according to claim 2, wherein the circuitry is configured to
calculate, based on the external condition data relating to the plurality of external data sources, a cost index and a quality index for each item identifier, and select, for each item included in the acquisition output list, a source that optimizes at least one of the cost index and the quality index.

4. The system according to claim 3, wherein the circuitry is configured to
include a result of the source selection in the prompt sentence to be input to the generative AI model so as to generate an optimized acquisition route.

5. The system according to claim 4, wherein the circuitry is configured to
transmit a fulfillment arrangement request to a transaction processing apparatus via the communication interface based on the acquisition output list and the external condition data.

6. The system according to claim 1, wherein the circuitry is configured to
periodically re-acquire image data from the imaging device, re-apply the object recognition model to generate updated object classification results and quantity estimates, and update stored item identifier and quantity data in a memory based on the updated results.

7. The system according to claim 6, wherein the object recognition model applies bounding box detection to the image data to estimate quantities, and is trained on a labeled dataset of item types and spatial configurations.

8. The system according to claim 1, wherein the wearable biological sensor device is configured to measure at least one of heart rate, body weight, physical activity level, and sleep pattern, and the time-series physiological data comprises measurements over a predetermined observation window.

9. The system according to claim 8, wherein the trend detection algorithm applies a sliding window analysis to the time-series physiological data to identify directional patterns, and the user state summary includes a trend classification and an associated confidence level.

10. The system according to claim 1, wherein the circuitry is configured to
apply emotion recognition processing to image data or audio data received from the terminal device, generate an emotion label based on the emotion recognition processing, and adjust at least one instruction parameter in the prompt sentence based on the emotion label.

11. The system according to claim 10, wherein the emotion recognition processing applies a convolutional neural network to the image data to detect facial expression features, or applies a recurrent neural network to audio features extracted from the audio data to generate the emotion label.

12. The system according to claim 1, wherein the circuitry is configured to
normalize the external condition data for each of the plurality of external data sources and calculate evaluation values for items corresponding to the object classification results prior to generating the constraint condition data.

13. The system according to claim 1, wherein the circuitry is configured to
record an execution log comprising the prompt sentence and the corresponding operational scheduling output and acquisition output list for each invocation of the generative AI model, and store the execution log in a memory.

14. The system according to claim 13, wherein the circuitry is configured to
retrieve a prior execution log when the constraint condition data or preference condition data are updated, compare the prior operational scheduling output against the updated operational scheduling output, and generate a change summary for transmission to the terminal device.

15. The system according to claim 1, wherein the constraint condition data comprises at least one of a numerical condition parameter, a time horizon parameter, and an exclusion condition derived from the user state summary and the object classification results.

16. The system according to claim 1, wherein the constraint violation detection algorithm compares each item identifier in the acquisition output list against the constraint condition data, and flags item identifiers that violate the constraint condition data prior to the correction step.

17. The system according to claim 16, wherein the circuitry is configured to
log flagged item identifiers and associated constraint violations in the memory, and include the logged constraint violations in the updated prompt sentence to reduce recurrence in subsequent regeneration iterations.

18. A system comprising:
circuitry configured to:
apply a convolutional neural network-based object recognition model to image data from an imaging device to generate object classification results and quantity estimates, and acquire time-series physiological data and behavioral data from a wearable biological sensor device via a communication interface;
apply a trend detection algorithm to the physiological data to generate a user state summary, generate constraint condition data and preference condition data by combining the object classification results, the user state summary, and external condition data, and encode the constraint condition data and preference condition data as a structured natural-language prompt sentence;
input the prompt sentence to a generative AI model to generate an operational scheduling output and an acquisition output list, parse the outputs into structured records, and apply a constraint violation detection algorithm to identify and remove or replace violating records; and
transmit the structured records to a terminal device, receive modification operations, update the constraint condition data and the prompt sentence based on the modification operations, and re-invoke the generative AI model to iteratively refine the operational scheduling output and the acquisition output list.

19. The system according to claim 18, wherein the circuitry is configured to
apply emotion recognition processing to sensor data from the terminal device to generate an emotion label, and adjust at least one instruction parameter in the prompt sentence based on the emotion label.

20. A method comprising:
applying a convolutional neural network-based object recognition model to image data captured by an imaging device to generate object classification results and quantity estimates for items in a monitored storage location, and acquiring time-series physiological data and behavioral data from a wearable biological sensor device via a communication interface coupled to a packet-switched network;
applying a trend detection algorithm to the time-series physiological data and behavioral data to generate a user state summary, and generating constraint condition data and preference condition data by combining the object classification results, the quantity estimates, the user state summary, and external condition data acquired from external data sources via the communication interface;
encoding the constraint condition data and the preference condition data as a structured natural-language prompt sentence and inputting the prompt sentence to a generative AI model to generate an operational scheduling output and an acquisition output list for a predetermined period;
tokenizing and parsing the operational scheduling output and the acquisition output list into structured records comprising item identifiers, quantity values, numerical parameters, and source identifiers, applying a constraint violation detection algorithm to each structured record to identify violations of the constraint condition data, and removing or replacing violating records; and
transmitting the structured records to a terminal device via the communication interface, receiving modification operations from the terminal device, updating the constraint condition data and the prompt sentence based on the modification operations, and re-invoking the generative AI model using the updated prompt sentence to iteratively refine the operational scheduling output and the acquisition output list.
